(19) United States
(12) Patent Application Publication
(10) Pub. No.: US 2022/0358646 A1
(43) Pub. Date: Nov. 10, 2022

(54) CELL ACTIVITY MACHINE LEARNING

(71) Applicant: Q-STATE BIOSCIENCES, INC., Cambridge, MA (US)

(72) Inventors: Steven Ryan, Cambridge, MA (US); Nathaniel Delaney-Busch, Cambridge, MA (US); Graham T. Dempsey, Sudbury, MA (US)

(21) Appl. No.: 17/735,799

(22) Filed: May 3, 2022

Related U.S. Application Data
(60) Provisional application No. 63/184,076, filed on May 4, 2021.

(51) Int. Cl. G06T 7/00; G01N 33/50; G06N 3/08

(52) U.S. Cl. CPC G06T 7/0012; G01N 33/5032; G06N 3/08; G06T 2207/30024; G06T 2207/20081; G06T 2207/20084; G06T 2207/10016; G06T 2207/10056

(57) ABSTRACT

The present invention provides methods and systems using optogenetic assays to identify features in measured neuronal activity that can be used to characterize neural disorders and potential therapeutic treatments.

### FIELD OF THE INVENTION

The invention generally relates to methods and systems for identifying therapeutic compounds.

### BACKGROUND

Significant resources have been devoted to understanding the causes, mechanisms of action, and potential treatments of neurological disorders. Despite the time and resources spent on understanding the mechanisms causing neurological disorders, the functional pathogenesis of many syndromes remains unknown. This provides an impediment to efficiently screening for potential therapeutics to treating neurological disorders.

The limited progress in neuroscience drug discovery is attributable, in part, to both a lack of translatable model systems and a lack of screening technologies with outputs predicting a primary therapeutic endpoint. For example, reliance on animal models in neuroscience drug discovery has led to a number of clinical disappointments due in part to lack of strong model validation. Rodent models have historically been poor predictors of efficacy in humans. In addition, animal models do not typically afford the throughput needed to screen compound libraries.

Perhaps more fundamentally, existing neurological models and screening modalities lack a way to effectively characterize neural disorders and drug responses in a manner that allows for comparisons across a number of tangible, defined measurements. Rather, most models and screening modalities must be designed around a particular disorder or drug, and their outputs provide minimal information relevant beyond a particular experiment.

### SUMMARY

The present invention provides for using optogenetic assays and machine learning systems to identify features or parameters in recorded action potentials from electrically excited cells. The identified features can be used to characterize the functional phenotype, or fingerprint, of both healthy and diseased cells, as well as to identify drugs that affect the phenotype.

Importantly, methods of the invention may be used for drug discovery for any disease by producing a result correlating to the therapeutic efficacy of a compound. This is accomplished by the nature of the novel machine learning system as disclosed herein. Specifically, the machine learning system may be trained using manually selected gene targets for any disease, and manually selected families of compounds that modulate the targets. In this way, a phenotype is developed for all the diseases the machine learning system has been trained on, which allows for drug discovery for any disease. Thus, methods of the invention enable fingerprinting compound effects and disease phenotypes relative to a control for any disease.

Methods and systems of the invention use optogenetic assays to provide detectable signals indicative of neuronal action potentials. Those signals are recorded over time, for example, as a video. Within these signals are hundreds of unique features or parameters of the action potentials. The invention uses machine learning systems and processes to identify, analyze, and select a statistically significant subset of these features/parameters. The subset of features is used to create a functional phenotype. The functional phenotype may be indicative of healthy cells or of a certain neuronal disorder. The functional phenotype may further be Thus, the invention identifies features of action potentials associated with neuropathologies.

When compared to the raw video signals from which they are derived, extracted action potential features are greatly reduced in terms of complexity. The resulting action potential data provide tangible measurements that characterize the effects of disorders and therapeutics on cell behavior with an unprecedented breadth, depth, and granularity.

Furthermore, reducing the optical signals from raw video, to action potentials (e.g., as voltage traces), action potential features and patterns, and finally, functional phenotypes, allows the systems and methods of the invention to significantly reduce the data footprint required to derive meaningful and multidimensional measurements of cellular behavior. In conjunction with data compression methods described below, this allows cellular behaviors to be efficiently stored and manipulated in a database, which allows high-throughput analyses of, for example, cell type, cell states, disease phenotype, and pharmacological response. Moreover, these phenotypes can be stored on a database as models, such as disease models, for comparison. This eliminates the need to reproduce labor- and reagent-intensive screens and experiments.

The invention also includes methods and systems to address and parse data generated when creating these functional phenotypes. The present Inventors discovered that a single instrument recording action potential signals during an 8-hour period generated over 50 terabytes of uncompressed, raw data. Methods of the invention may overcome this hurdle while using a lossy compression scheme that compresses the data by a factor of between 20× and 200×. Moreover, despite the lossy nature of the compression, there is little or no loss of critical data. In fact, in certain instances, only undesirable artifacts in the data were lost during compression.

By using machine learning systems, systems and methods of the invention generate functional phenotypes for disease cells, which reveal the salient differences in action potential features when compared to healthy cells. These differences characterize the behavior of disease cells and can be used as a direct comparison to a test cell for diagnostic purposes. Correlating these differences with a neuropathology (via associated symptomology, other diagnostic tests and the like) allows the functional phenotypes to be used as diagnostic criterion. Moreover, the identified differences in features provide meaningful targets used, for example, to conduct subsequent drug screens or any appropriate informatic purpose. The systems and methods of the invention can likewise create functional phenotypes revealing changes in cell behavior caused by administering a known or potential therapeutic. Advantageously, this core concept can be expanded, for example, to identify potential therapeutic treatments for a disorder, to predict potential side effects of drug candidates, identify candidate treatments with reduced or no side effects compared with extant treatments, synergistic or combination treatments using multiple compounds, and even to quickly screen known compounds for potential second treatment uses.

In certain aspects, the disclosure provides methods for characterizing cellular activity. The methods include making a recording of activity of one or more electrically-active cells, presenting the recording to a machine learning system trained on training data comprising recordings from cells with a known pathology and cells without the pathology, and reporting—by the machine learning system—a phenotype of the electrically-active cells. The recording may comprise one or more action potentials exhibited by the electrically-active cells. The machine learning system reports the phenotype of the electrically-active cells as e.g. having or not having the pathology. The method may include exposing the electrically-active cells to a test compound. The machine learning system may report the phenotype of the electrically active cells as reverting from having the pathology to not having the pathology with exposure to test compound. Preferably the recording is a digital movie made by imaging the electrically-active cells through a microscope with a CMOS images sensor. The machine learning system is resident in a computer system comprising a processor coupled to memory, and the recording is saved in the memory.

In some embodiments, the recording captures action potentials, and the method further comprises measuring, and storing, a plurality of features from the action potentials. The method may include measuring features from the recording and presenting the features to the machine learning system, optionally wherein the features comprise one or more of spike rate, spike height, spike width, depth of afterhyperpolarization, onset timing, timing of cessation of firing, inter-spike interval, adaptation over a constant stimulation, a first derivative of spike waveform, and a second derivative of spike waveform. The method may include operating the machine learning system under control of a budget wrapper that limits a number of features that are presented to the machine learning system. The method may include extracting greater than hundreds features from the recording and the budget wrapper may presents fewer than about a dozen or so of the features to the machine learning system.

In certain embodiments, the machine learning system comprises a neural network. The neural network may an autoencoder neural network that operates by representation learning. Preferably, the autoencoder has been trained using manually selected training data comprising the recordings from cells with the known pathology and the cells without the pathology in samples that have been exposed to known compounds with known efficacy and control samples that have not be exposed to the known compounds.

Some embodiments use a bootstrapping algorithm to create augmented data for a training data set. Because some deep learning methods are prone to overfitting to the training data, in embodiments, methods of the invention use a bootstrapping algorithm to provide augmented training data, useful to avoid a machine learning system prone to overfitting. Prior art data augmentation methods have addressed overfitting by injecting noise into existing data or parameterizing the characteristics of the data set in order to generate similar synthetic data. In contrast, methods of the invention use bootstrapping to resample (e.g., with replacement) from within the training data to create augmented data without any requirement for synthetic data.

Other aspects provide methods for compressing raw movie data. Methods include obtaining digital video data of electrically active cells and processing the video data in a block-wise manner by, for each block, calculating a covariance matrix and an eigenvalue decomposition of that block and truncating the eigenvalue decomposition and retaining only a number of principal components, thereby discarding noise from the block. The video is written to memory as a compressed video using only the retained principal components.

The blocks may be selected by parcellating the data using region-based tiling based on a local intensity maxima of a mean movie frame. The digital video data may be obtained from electrically active cells expressing optical reporters of cellular electrical activity. In certain embodiments, the cells are neurons and the digital video data shows action potentials propagating along axons of the neurons. Preferably, the compressed video can be retrieved and played to display the action potentials propagating along the axons of the neurons.

The method may include measuring, by a machine learning system, features from the action potentials, wherein the machine learning system obtains the same values for the measured features whether measuring from the digital video data or the compressed video.

In preferred embodiments, the compressed video occupies less than about ten percent of disc space required for the digital video data. The obtaining step may include filming, through a microscope and using a digital image sensor, live neurons firing. Preferably the digital image sensor is connected to a computer that performs the processing step, and the compressed video may be written to a remote computer via an Internet connection. In some embodiments, the digital image sensor produces over fifty terabytes of the digital video data in one day. The processing step may compress the digital video data by at least about twenty times.

In other aspects, the invention provides methods using machine learning to characterize a cellular behavior based on features measured from action potentials. An exemplary method for characterizing a neural phenotype includes recording action potentials of stimulated neural cells with a known pathology and stimulated neural cells without the pathology. Features of said action potentials associated with the pathology are identified and used to train a machine learning system. The machine learning system then generates a functional phenotype using a subset of the action potential features to characterize the pathology. The machine learning system may be subject to constraints on the desired dimensionality or information utilization of the phenotype, and the machine learning system may search for optimal phenotype representations under those constraints. This reduces the dimensionality of the phenotype, which can reduce its data size, limit the phenotype to significant features, and provide an approachable measurement for cellular behavior. In exemplary methods of the invention, the machine learning system learns and/or identifies a plurality of features. In such methods, a budget wrapper restricts the input to the machine learning system to fewer than about, e.g., 12 features to generate the functional phenotype. In exemplary methods of the invention, the machine learning system estimates a reasonable budget from the data (which may be tens to hundreds of features), then finds the optimal combination of features that best discriminate healthy from diseased cells given the budget constraints. The optimal combination may include any single feature, a plurality of features, or all available features. The features selected by the machine learning model under these conditions are then evaluated for statistical significance in an independent sample.

The machine learning system may use functional phenotypes generated for a particular pathology to provide an output identifying one or more of the learned action potential features as a target for treating the pathology.

The present invention also provides an exemplary method for assessing a cellular pathology that includes obtaining neural cells having a known pathology and causing the cells to express optical reporters of membrane electrical potential. Then, the method includes stimulating the neural cells in wells of a multi-well plate such that they exhibit action potentials. Optical signals from the optical reporters, in response to the stimulated action potential, are recorded. Action potential features are identified from the recorded optical signals.

The invention also provides methods for diagnosing a pathology using functional phenotypes. In an exemplary method, action potential features from a test neural cell are identified and used by a machine learning system to generate a functional phenotype for the test cell. The test neural cell can be obtained or derived from a sample from a subject. The method then includes determining whether the test neural cell has the pathology based upon the extent to which the test neural cell phenotype matches that of the neural cell with the pathology. The method can then provide a diagnosis, which can be a score of reduced dimensionality relative to the functional phenotypes.

The present invention also provides methods and systems for assessing efficacy of a drug against a neuronal pathology using a machine learning system to generate functional phenotypes. An exemplary method for assessing efficacy includes measuring action potentials of neurons exposed to a known therapeutic compound and identifying features of the action potentials associated with therapeutic efficacy. A machine learning system is trained using these features such that it can assess the therapeutic efficacy of a test compound.

The machine learning system may be operated under the control of a budget wrapper. For example, when assessing drug efficacy, the machine learning system may be exposed to a plurality of features where the budget wrapper selects a subset of the features.

In exemplary methods for assessing drug efficacy, the measured action potentials are from neurons of a specific pathology and the machine learning system provides an output identifying the learned action potential features as targets for treating the pathology.

In certain methods of the disclosure the functional phenotypes are validated, for example, using hierarchical bootstrapping. Such methods can include resampling from the training data at each relevant level of the sampling hierarchy to detect or avoid effects of intra-class correlation within a plurality of in vitro assays.

Exemplary action potential features identified from the recording include, for example, one or more of fluorescence, spike height, width, shape change, slope, frequency, timing, refraction, bursting, synchrony, and relationship to stimulation.

Embodiments of the disclosure provide compression algorithms that compress movies, particularly useful for neural imaging movies (e.g., calcium imaging or optogenetic movies). Some embodiments compress the recorded signals using a lossy algorithm. The lossy algorithm may include a principal component analysis (PCA), such as a patchwise PCA. In an exemplary method, the lossy algorithm compresses the recorded signals by a factor of at least 20×. Advantageously, the lossy algorithm primarily loses unwanted noise from the recorded signals.

In exemplary systems and methods of the invention, a machine learning system identifies spatiotemporally correlated optical signals in each well of the plate to associate optical signals with certain cells in each well.

In certain aspects, the invention provides a method for drug discovery. The method includes exposing electrically-excitable cells to a compound, measuring the electrical activity of the cells, measuring features of action potential of the cells, and using a machine learning system to assess therapeutic efficacy of the compound based on the input measured features.

As noted, the action potential features may be identified for a single cell and include one or more of spike rate, spike height, spike width, depth of afterhyperpolarization, timing of spike onset, timing of cessation of firing, an inter-spike interval of a first spike, extent of adaptation over a constant stimulation, a first derivative of spike waveform, and a second derivative of spike waveform. The machine learning system is trained to identify features of electrical activity associated with the therapeutic efficacy of a compound.

Because the features identified may be an output of tabular data with non-linear relationships between measures, the machine learning system may comprise an autoencoder neural network as described above. The autoencoder may essentially be a representation-learning algorithm configured to map raw measurements onto a biological representation. Importantly, the autoencoder may be trained using manually selected gene targets and manually selected compounds that modulate the targets. The autoencoder may further be trained using hyperparameter tuning by optimizing the depth, width, nonlinearities, batch size, learning rate, momentum, gradient clipping, and training cycles of the autoencoder. These tuned hyperparameters have a large influence on model performance and utility.

In embodiments, methods of the invention provide for detecting activity in compounds. It is valuable to know which biological samples contain compounds showing signs of activity. For example, finding biologically active compounds in a screen, or finding the lowest dose with detectable activity.

### BRIEF DESCRIPTION OF THE DRAWINGS

FIG. 1 provides voltage traces measured from an optical reporter of membrane potential.

FIG. 2 shows features that can be identified using optical reporters.

FIG. 3 compares voltage traces from wildtype (WT) and knockout (KO) neurons.

FIG. 4 provides a radar plot of features in control cells (e.g., WT) and diseased cells.

FIG. 5 provides a radar plot of action potential features.

FIG. 6 shows a functional phenotype from an in silico model of disease cells.

FIG. 7 provides two exemplary radar plots representing functional phenotypes.

FIG. 8 shows features mapped onto a lower-dimensional space.

FIG. 9 shows phenotype reversal (x-axis) and side effects (y-axis) for two compounds.

FIG. 10 shows features, ranked by importance, measured from action potentials.

FIG. 11 shows providing sparsity to a machine learning system model.

FIG. 12 shows components of an exemplary microscope.

FIG. 13 shows a prism that guides the beam towards a sample.

FIG. 14 shows an optical light patterning system for a microscope.

FIG. 15 shows an overlay for hiPSC-derived neurons identified by automated analysis.

FIG. 16 demonstrate the underlying variability in neuronal behavior.

FIG. 17 provides a raster plot where each point is an identified action potential.

FIG. 18 provides the spike rate averaged over the cells (the firing rate).

FIG. 19 provides spike shape parameters extracted from the action potentials.

FIG. 20 provides spike timing parameters extracted from the action potentials.

FIG. 21 provides the adaptation average over the cells.

FIG. 22 shows the clear reduction in neuronal excitability caused by ML-213.

FIG. 23 shows functional phenotypes generated by a machine learning system.

FIG. 24 shows phenotype reversal.

FIG. 25 have increasing effects on KO cell behavior as the concentration increases.

FIG. 26 shows drug-induced changes in neuronal spiking.

FIG. 27 provides concentration response curves for varied concentrations of compounds.

FIG. 28 shows high SNR fluorescent voltage recordings.

FIG. 29 shows a raster plot showing spikes recorded in each column.

FIG. 30 provides the average firing rate during stimulus.

FIG. 31 provides a heat map showing the number of spikes.

FIG. 32 provides a plot of the average number of spikes.

FIG. 33 provides a plot of the average number of spikes recorded for DRG neurons.

FIG. 34 shows elimination of a protein in knockout cells.

FIG. 35 provides a spike from voltage traces recorded across multiple cell lines.

FIG. 36 provides a spike from voltage traces.

FIG. 37 provides a multidimensional radar plot with a functional phenotype.

FIG. 38 provides a disease score that represents a further dimensionality reduction.

FIG. 39 provides spike parameters and spike rates.

FIG. 40 shows CheRiff expressed in a subset of neurons.

FIG. 41 provides a fluorescence image obtained using a microscope.

FIG. 42 shows fluorescent traces showing postsynaptic potentials (PSPs).

FIG. 43 shows modulation of single cell PSPs in response agonists.

FIG. 44 shows average PSP traces for control pharmacology.

FIG. 45 plots drug-induced change normalized to the mean pre-drug response.

FIG. 46 diagrams an exemplary method for high-throughput screening.

FIG. 47 shows a computer system makes a recording of electrically-active cells.

FIG. 48 diagrams a recursive resampling routine.

### DETAILED DESCRIPTION

The present invention provides methods and systems using optogenetic assays and machine learning to identify features or parameters in recorded action potentials from electrically excited cells, which can be used to characterize neural disorders by functional phenotype. In preferred embodiments, a machine learning system is trained using data sets of action potential measurements associated with, for example, cells with a known pathology and healthy cells. The machine learning system identifies features of action potentials and uses a subset of those features to generate a functional phenotype that reveals the differences in cellular behavior in healthy/control cells compared to diseased cells, cells exposed to a certain compounds or environmental conditions, and different cell types.

In optogenetics, light is used to control and observe certain events within living cells. For example, a fluorophore-encoding gene, such as a fluorescent voltage reporter, is introduced into a cell. The reporter may be, for example, a transmembrane protein that generates an optical signal in response to changes in membrane potential, thereby functioning as an optical reporter. When excited with a stimulation light at a certain wavelength, the reporter is energized to and produces an emission light of a different wavelength, which indicates a change in membrane potential. Cells in the sample may also include optogenetic actuators, such as light-gated ion channels. Such channels respond to a stimulation light of a particular wavelength, leading to changes in cellular activity, including the generation of action potentials or post-synaptic potentials. Methods and systems of the invention may use additional reporters of cellular activity, and the associated systems for actuating them. For example, proteins that report changes in intracellular calcium, intracellular metabolite or second messenger levels.

In an exemplary method, gene editing techniques (e.g., use of transcription activator-like effector nucleases (TALENs), the CRISPR/Cas system, zinc finger domains) are used to create a cell that is isogenic but for a variant of interest. The cell is converted into an electrically excitable cell such as a neuron or cardiomyocyte. The cell may be converted to a specific neural subtype (e.g., motor neuron). The cell is caused to express an optical reporter of a cellular electrical activity, which emits a fluorescent signal in response to changes in the cellular membrane potential when the cell exhibits an action potential. The cell may also be caused to express an optical actuator of cellular activity, which causes activity in the cell upon activation by light.

The cell is stimulated, e.g., through optical, synaptic, chemical, or electrical actuation. In response to the stimulus, the cell may exhibit an action potential. Using microscopy and analytical methods described herein, the response of the cell to the stimulus is measured using a fluorescent signal from the optical reporter. The signal from the optical reporter varies in response to changes in the cell's membrane potential, which is indicative of an action potential caused by the stimulation.

Features or parameters in the detectable fluorescent signal are then identified. In certain methods and systems of the disclosure, automated algorithms, including machine learning and signal processing, are used to identify features or parameters of an action potential in the signal.

Measurements may be made over time for neural cells expressing optical reporters of membrane potential. The cells may express optical actuators of a cellular activity. A stimulus light directed onto the cells actuates the actuators, which leads to a change in membrane potential. The stimulus light can be transmitted to the cells in pulses of varying or ramped intensity or frequency. The measurements (voltage traces) show spikes in the fluorescent signal generated by the reporter. Each spike is an action potential caused by exposure to the stimulus.

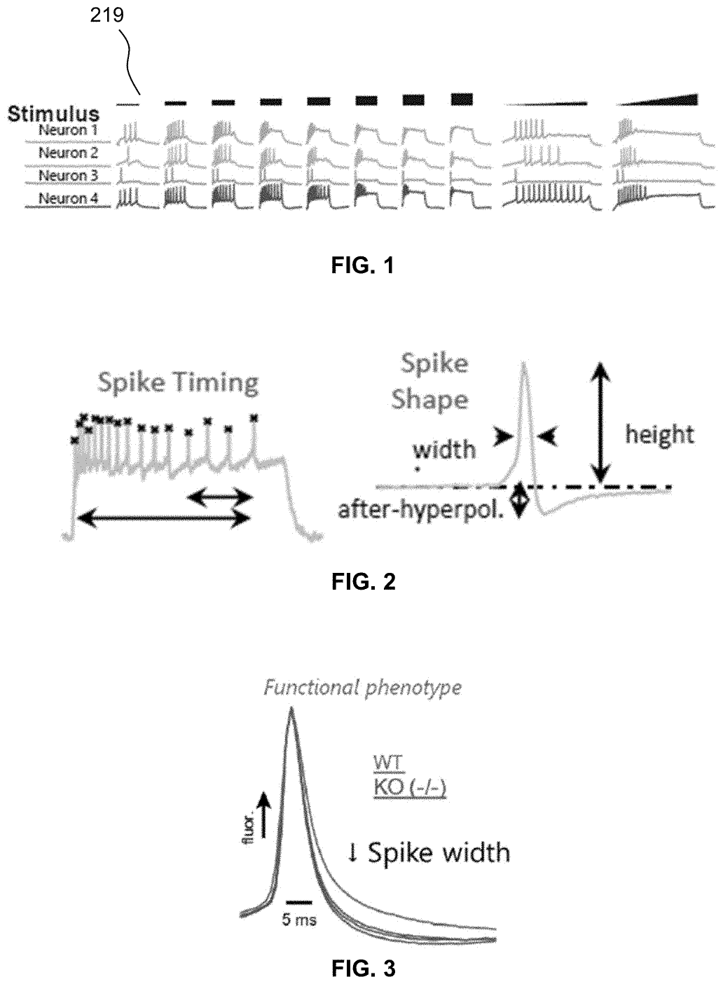

FIG. 1 provides exemplary voltage traces measured from an optical reporter of membrane potential. In the systems and methods of the invention, action potential features are identified from these voltage traces. A machine learning system can be trained and used to identify action potential features from voltage traces.

FIG. 2 provides limited examples of features or parameters that can be identified in the signals from the optical reporters. As shown, the features such as spike timing, shape, width, frequency, and height can be identified in the signals. Systems and methods may identify at least 300 individual and unique action potential features in the signals measured from the optical reporters. These features can be used by a machine learning system to generate a phenotype that characterizes a particular neuropathology.

The system may impose constraints on the desired dimensionality or information utilization of the phenotype, and searche for optimal phenotype representations under those constraints. This reduces the dimensionality of the phenotype, which can reduce its data size, limit the phenotype to significant features, and provide an approachable measurement for cellular behavior. In exemplary methods of the invention, the system measures and/or identifies a plurality of discernable features. In such methods, a budget wrapper requires, for example, a machine learning system to receive only a subset of features to generate the functional phenotype.

FIG. 3 shows a comparison of partial voltage traces recorded from wildtype neurons (“WT”) and a neuron with a knockout mutation (“KO”) that models a particular neural disorder. In this instance, the KO cells show an action potential feature of a reduced spike width on the voltage trace when compared to the WT cells. A computer system may measure hundreds or more unique action potential features from a trace. Preferably, only a subset of those features are provided as input to a machine learning system to generate a functional phenotype that characterizes the behavior of the cells. The machine learning system may be trained using data sets of action potentials or action potential features associated with either healthy/control cells or cells with, or that model, a particular neural condition. The differences in the identified action potential features between healthy or wildtype cells and cells with a neural disorder provides a functional phenotype for the disorder. The system can select a subset of these features from which it generates the functional phenotype. A similar comparison can be done, for example, using any of the individual action potential features and in cells exposed to different therapeutic compounds, stimuli, environmental conditions, etc.

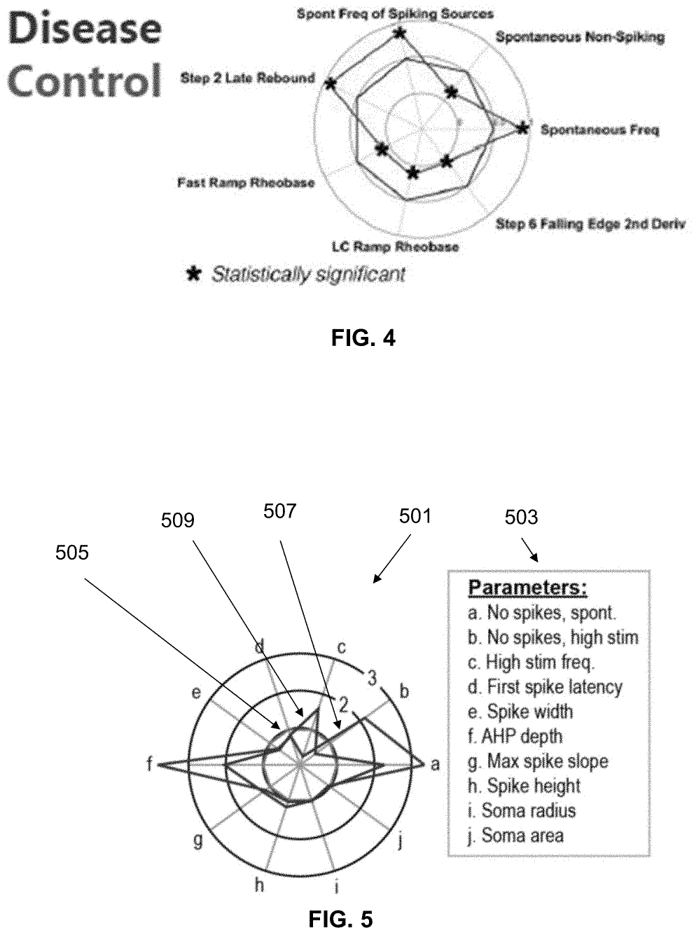

FIG. 4 provides a radar plot of action potential features measured and identified in control cells (e.g., the WT neurons) and diseased cells (e.g., the KO neurons), which provides a representation of a functional phenotype generated by a machine learning system of the invention. The values for each action potential feature are normalized to the values of the control cells. From among all action potential features identified, the plotted action potential features are determined by the system to be predictive in this comparison. The differences among plotted features provide the functional phenotype of the disease, which characterizes the neuron's behavior in response to the disease.

Advantageously, the functional phenotypes, action potential measurements, and action potential features of the control cells can be stored on a relational database, where they provide an in silico model of the control cells. Subsequent action potentials measured from different stimulated cells can be compared with this in silico model using the machine learning system to generate a functional phenotype.

FIG. 5 provides an exemplary radar plot 501 of action potential features/parameters 503 identified for a control cell 505, e.g., a wildtype cell population. The plot for the control cells may be from an in silico model of the control on a rational database. The plot 501 also provides the same features for a first cell population 507 and a different second cell population 509. The magnitudes of the measured features are normalized to the values measured of the control cells. The first and second cell populations are, for example, cells with different neural disorders. The plotted differences in the action potential features of the first and second cell populations when compared with those of the control represent the functional phenotypes for the first and second cell populations.

As shown more than two phenotypes can be overlayed, for example, to compare various cell types, responses to compounds, therapeutic efficacies and side effects, different neural conditions, and the like. In some applications, it is valuable to measure different cell populations simultaneously from the same preparation. For each cell measured, its membership in one subpopulation or another can be determined either at the time of Optopatch recording or later using other methods. This enables the systems and methods of the invention to, for example: identify discrete therapeutically relevant subpopulations in a single cell preparation; eliminate idiosyncratic variance between cell culture preparations when comparing populations; or investigate complex interactions in heterogeneous populations of wildtype and disease neurons. Optopatch, recording action potentials, neurons, optogenetics, and other features of this disclosure may use any of the elements, methods, and features shown in any one or any combination of U.S. Pat. Nos. 9,057,734; 9,207,237; 9,594,075; 9,518,103; U.S. patent Ser. No. 10/048,275; U.S. patent Ser. No. 10/392,426; U.S. patent Ser. No. 10/457,715; U.S. patent Ser. No. 10/107,796; U.S. patent Ser. No. 10/161,937; U.S. patent Ser. No. 10/352,945; U.S. patent Ser. No. 10/288,863; and U.S. patent Ser. No. 10/613,079, all incorporated by reference, for all purposes.

First 507 and second 509 cells may, for example, be cells exposed to different compounds, stimuli, environmental conditions, etc. The control cells may be, for example, wildtype cells or derived from wildtype cells. The control cells may also be cells with a particular disorder, such a neural disorder, cells modeling disorder, cells with a particular mutation, and the like.

Like the control, the functional phenotype, action potential measurements, and action potential features of the first 507 and second 509 cells can be placed on a relational database to provide in silico models.

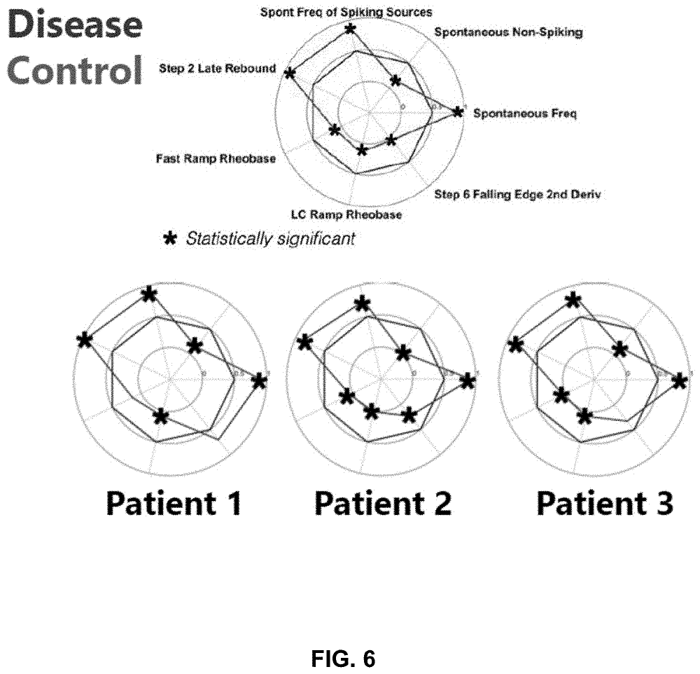

FIG. 6 shows a functional phenotype from an in silico model of disease cells, which could be placed on a relational database and used for diagnostic purposes. Cells may be obtained or derived from cells of a patient. The functional phenotypes generated for these cells can be compared to that of the disease cell model. If the phenotypes overlap sufficiently, for example as defined by a particular threshold or disease score, a diagnostic, or a prophylactic can be provided.

The present invention also provides methods and systems for assessing efficacy of a drug against a neuronal pathology using a machine learning system to generate functional phenotypes. An exemplary method for assessing efficacy includes measuring action potentials of neurons exposed to a known therapeutic compound and identifying features of the action potentials associated with therapeutic efficacy. A machine learning system is trained using these features such that it can assess the therapeutic efficacy of a test compound.

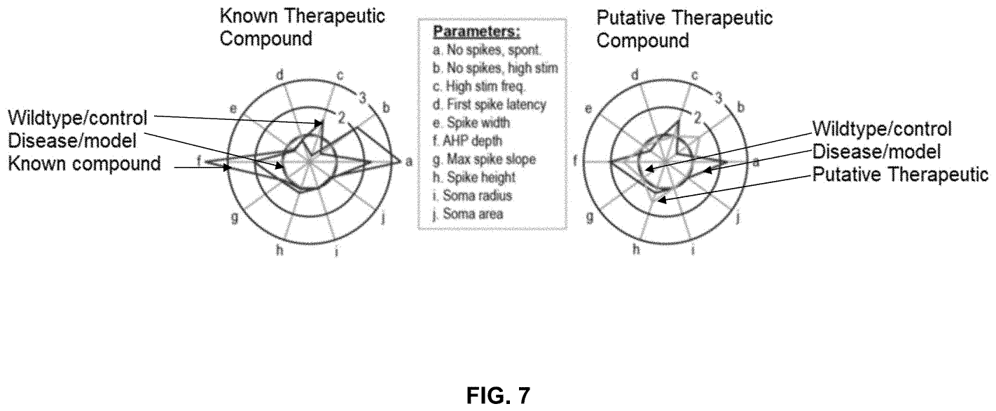

FIG. 7 provides two exemplary radar plots representing functional phenotypes generated from action potential features using a machine learning system. The “known therapeutic” plot includes a subset of measured action potential features identified by the machine learning system for: stimulated wildtype/control cells (e.g., neurons expression an optical reporter); stimulated cells from a subject with a neural disorder or disease or cells that model a disorder; and the disease/model cells stimulated in the presence of a known therapeutic. The magnitudes of the identified action potential features are normalized to the values measured for the wildtype/control cells. The unique action potential features identified in the cells stimulated in the presence of the known therapeutic are the functional phenotype of the therapeutic, i.e., the change in cellular behavior caused by contact with the therapeutic. The functional phenotype can thus be correlated with the therapeutic's efficacy in treating a particular neural disorder or disease. Unique action potential features may be identified in the cells stimulated in the presence of the therapeutic that converge with the values for those same features in a wildtype/control cells can be correlated with a therapeutic benefit. The features that diverge from those of the wildtype/control can be correlated with a potential side effect of the known therapeutic. The “putative therapeutic” plot provides a subset of identified measured action potential features/parameters for stimulated wildtype/control cells, stimulated cells from a subject with a neural disorder or disease or cells that model a disorder, and the disease/model cells stimulated in the presence of a putative therapeutic.

In an exemplary method, after measured action potential features are identified for a cell in the presence of the putative therapeutic compound the machine learning system assesses the therapeutic efficacy of the putative therapeutic by mapping the features against substantially identical features present in stimulated cells treated with one or more compound known to be efficacious in treating a neuronal disease. In some embodiments, ˜300 features/parameters are identified and mapped onto a ˜300 dimensional space as vectors. The vectors thus describe the disease phenotype and/or compound effects on the cells as indicated by the measured action potential features.

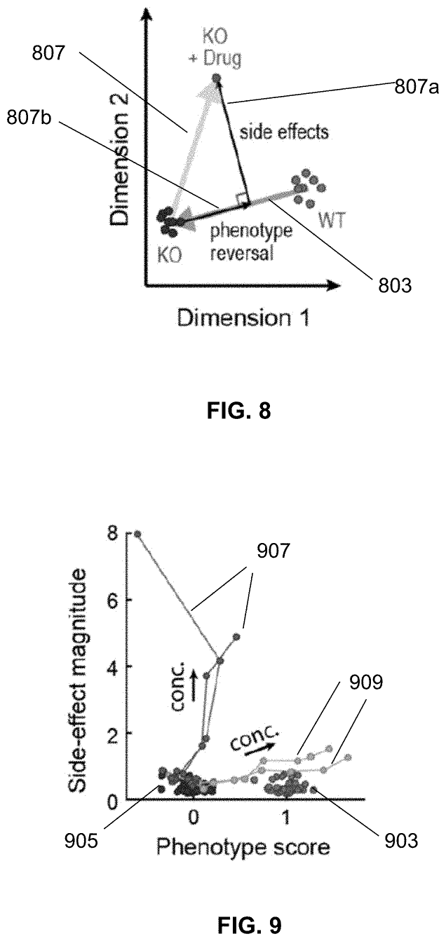

FIG. 8 provides an example of identified features mapped onto a lower-dimensional space. For clarity, the map only shows two dimensions that each correspond to a unique action potential feature or group of features, i.e. a functional phenotype. Wildtype/control cells and disease/model cells can be separated into distinct groupings based on divergent values for their shared features. Vector 803 represents a reversal of the disease (or modeled disease) phenotype. Vector 807 represents the features/parameters caused by stimulating the disease cells in the presence of the compound, i.e., compound or drug effects. As shown, vector 807 is deconstructed into two separate component vectors, 807a and 807b. A component vector falls along the phenotype reversal vector 803 and represents the effect the compound has on reversing the disease/model phenotype (a therapeutic benefit). Component vector 807b is orthogonal to component vector 807a and represents effects of the compound that do not reverse the disease/model phenotype, and thus represents potential side effects.

FIG. 9 shows an example where the magnitudes of the phenotype reversal (x-axis) and side effects vectors (y-axis) are plotted for two putative therapeutic compounds. As in FIG. 8, wildtype/control cells are clustered 903 together based on shared action potential features. Similarly, the disease/model cells are clustered 905 together. Identified action potential features for the cells stimulated in the presence of a putative therapeutic compound are mapped 907 against substantially identical features 909 for cells stimulated in the presence of a known therapeutic compound. In this map, the magnitude of the identified features increases in magnitude in response to increasing concentrations of either the putative or known therapeutics.

The machine learning system thus predicts therapeutic efficacy of the putative therapeutic compound based upon the extent to which the functional phenotype for the putative therapeutic matches that of a known therapeutic compound. Because the mapped identified features for the putative therapeutic diverge from the substantially identical features for the known therapeutic, the predicted therapeutic effect of the putative therapeutic will be low. Further, a divergence between the identified features of the putative therapeutic and the substantially identical features of the known therapeutic may indicate a potential for the putative therapeutic to cause side effects. The machine learning system can be trained to recognize these divergent effects and provide a predictive output of potential side effects caused by the compound.

Advantageously, the exemplary method uses features/parameters associated with a known efficacious compound to derive the predicted efficacy for a putative therapeutic. Thus, even if the efficacious compound and the putative therapeutic have no indicated commonalities, e.g., structural similarities or common clinical indications, a prediction can still be derived. Further, there is no need for a priori information about how either compound achieves an effect in a cell. Rather, the change in cellular behavior caused by the compounds is used, as indicated in the action potential features, provides the basis for comparison.

In the methods described herein, action potential features associated with therapeutic efficacy may be derived from identifying action potential features of neurons exposed to a compound with a known efficacy in treating the neuronal disease. Alternatively or additionally, the features can be identified by comparing action potential features of neurons with and without the neural disease. Similarly, a comparison can be made between wildtype/control neurons and cells that model the disease phenotype. Models may include, for example, knock-in or knockout mutations that cause the disease phenotype. Alternatively or additionally, models may include actuators of cellular activity that, when actuated, cause the disease phenotype or rescue the neuron from the diseased state. Mapping the action potential features of the diseased neurons and healthy cells provides a phenotype for the disease, which can be described using a vector on a multidimensional space. The features can be stored, for example, in tabular form or a relational database such that for every compound tested, the features associated with therapeutic efficacy do not have to be re-identified. Compounds that induce action potential features that reverse this phenotype can be identified as putative therapeutics.

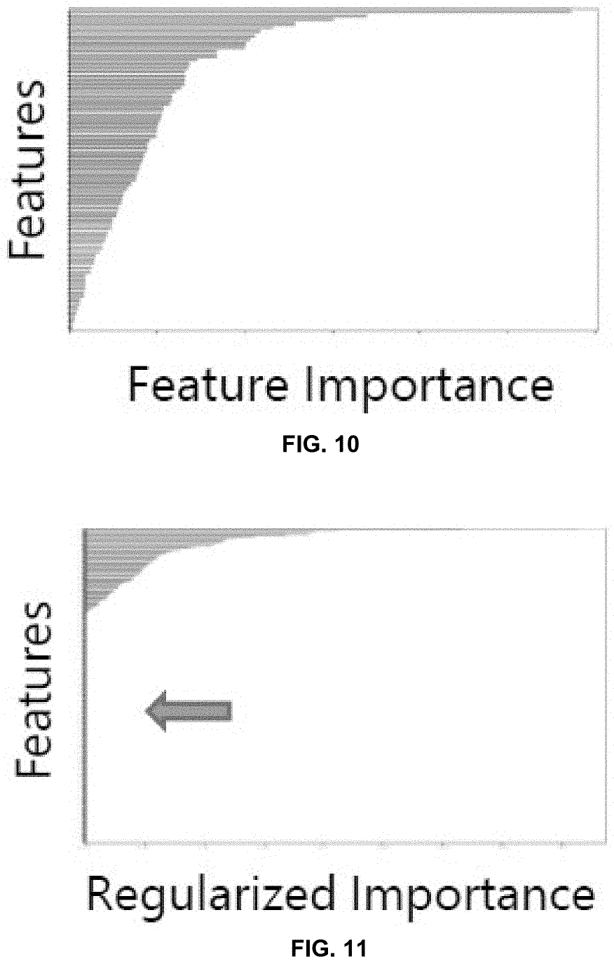

FIG. 10 shows features, ranked by importance, that may be measured from action potentials. A computer may measure many unique and discernable action potential features. Generally, the systems and methods identify around 300 or more features. In some embodiments, ˜300 features/parameters are identified and mapped onto a ˜300 dimensional space as vectors for control cells and test cells. The vectors of action potential features between control and test cells represents a phenotype and provide the functional phenotype of the test cells. The vectors thus characterize cellular behavior using features from measured action potentials.

In the methods and systems of the invention, a machine learning system is used to analyze action potential features to generate functional phenotypes for cells. By way of explanation, machine learning is a branch of artificial intelligence and computer science which focuses on the use of data and computer algorithms Machine learning is the study of computer algorithms that can improve automatically through experience and by the use of data. Machine learning algorithms build a model based on sample data, known as training data, in order to make predictions or decisions without being explicitly programmed to do so. Generally, machine learning systems of the invention identify a subset or composite of key action potential features, which are used to generate the functional phenotype. The machine learning system determines the relative importance of action potential features in their ability to establish a functional phenotype from the features. The machine learning system model can be validated or trained using a variety of methods.

Preferred embodiments of the machine learning system and associated algorithms are described in detail below. However, any of several suitable types of machine learning algorithms may be used for one or more steps of the disclosed methods and systems. Suitable machine learning types may include neural networks, decision tree learning such as random forests, support vector machines (SVMs), association rule learning, inductive logic programming, regression analysis, clustering, Bayesian networks, reinforcement learning, metric learning, manifold learning, elastic nets, and genetic algorithms. One or more of the machine learning types or models may be used to complete any or all of the method steps described herein. For example, in embodiments, the machine learning system may use one or more of random forest and shapely values, elastic net classifiers, y-aware principal component analysis (PCA), and hierarchical linear mixed effects models to identify high-information action potential features and/or generate functional phenotypes. As described below, in embodiments, the machine learning system utilizes novel algorithms for nested data to fully leverage this structure and to build powerful and efficient custom tools for in vitro biology applications.

In preferred embodiments, the machine learning system uses novel algorithms to derive drug fingerprints. As disclosed herein, methods of the invention capture electrophysiological measurements of each neuron, such as spike rate, spike height and width, the depth of the afterhyperpolarization, the timing of spike onset and cessation of firing, the inter-spike interval of the first spikes, the extent of adaptation over a constant stimulation, and first and second derivatives of the spike waveform. Stable patterns are apparent across measurements and across stimulation regimes within measurements. As examples, “fast action potential kinetics” alter nearly all measures of spike shape, and firing rate tends to increase with stimulation up to some maximal point, tracing a characteristic “frequency-intensity” curve. These complex, nonlinear, multidimensional patterns offer unique signatures of disease states and compound effects.

However, the large number of measurements—several hundred measurements from each cell—may be challenging as-is for downstream uses because of the high dimensionality of the data set. Dimensionality refers to how many attributes a data set has. High-dimensional data describes a data set in which the number of dimensions may be staggeringly high, as is the case in the instant invention, such that calculations can become extremely difficult. With high dimensional data, the number of features may far exceed the number of observations. High-dimensional readouts tend to perform poorly in many clustering, matching, and classification tasks, because high-dimensional spaces are sparse and most vectors are orthogonal. Some embodiments reduce a total number of features to a limited subset, a smaller number of the features, that are actually presented to the machine learning system.

FIG. 11 shows the application of regularization techniques to deliberately provide sparsity to the machine learning system model, i.e., to limit a number of features that are used as input to a machine learning system such as a neural network. For action potential from cells, e.g., associated with a particular pathology, features may be measured. A regularized importance may be assigned to features, and fewer than all measured features may be used as inputs to the machine learning system. In certain embodiments, a second (“pre-amp”) machine learning system is trained to evaluate importance of all features, and assigned a regularized importance to the features to provide output that assigns the regularized importance to all of the measured features. That allows a budge wrapper to select only a limited number (e.g., 12) of the features. By whatever method the features are selected, they form part of the input to the primary (“power amp”) machine learning system that is trained to give a cell phenotype and thus can show that a test drug has efficacy on a cell associated with a known pathology.

Preferably, the primary machine learning system is trained to distinguish action potential features of a healthy/control cell and a disease cell. The model is inspected to identify which features it identified as key to generating a functional phenotype characterizing the behavior the disease cell relative to the healthy cell. Inferential statistics, e.g., multilevel models, are also used to identify which features are a part of the functional phenotype. Both the machine learning and statistical models can be evaluated on additional data, e.g., holdout data.

Some embodiments use a machine learning system comprising an autoencoder neural network. An autoencoder is a type of artificial neural network used to learn efficient codings of unlabeled data and is understood to be an unsupervised learning technique. The autoencoder serves as a processing step for the machine learning system that encodes the data to be usable by the machine learning system. Autoencoders push information through a series of nonlinear transforms flowing through a low-dimensional bottleneck, and then try to reconstruct the raw data on the other side of the bottleneck. However, methods of the invention use the hypothesis that many high-dimensional data sets lie along low-dimensional manifolds inside that high-dimensional space. Thus, because the data measurements are often highly correlated, the high-dimensional raw data is highly concentrated along a lower-dimensional nonlinear manifold, such that the data set can be described using a comparatively smaller number of variables.

In embodiments, the autoencoder neural network is trained on a data set of diverse compound signatures for the purpose of finding the lower-dimensional nonlinear manifold that correlates to the high-dimensional raw data. This approach allows the autoencoder to discover the representations required for feature detection and classification from the raw data. The dimensions of this manifold each pertain to different patterns of activity in the underlying biology. Thus, the autoencoder effectively acts as a representation-learning algorithm, capable of mapping raw measurements onto biological representations.

The success of this approach is achieved by the nature of the training data set used for the purpose of constructing a coherent fingerprint. The behavior and utility of the autoencoder is largely a function of the training data used. In some embodiments, training data is created by first sequencing the RNA from neural preparations to find the gene targets of interest. Targets are selected to represent a diverse range of diseases and conditions. Compounds that selectively modulate the targets—both activators and blockers—are then manually identified. Data for the compounds is collected, including, in embodiments, a 10-point dose response, in quadruplicate, with an imaging protocol as disclosed herein to maximize the information extracted from each neuron. This results in a data set of highly active compounds, across a range of activity levels, for many different classes of compounds. This type of data set requires the autoencoder to encode a very diverse set of fingerprints for compounds that radiate out from a central cloud of inertness like rays from the sun, moving further from the center as the dose increases. Additionally, the depth, width, nonlinearities, batch size, learning rate, momentum, gradient clipping, and training cycles of the autoencoder for these data are optimized. These tuned hyperparameters have a large influence on model performance and utility.

The raw measurements are adjusted using hierarchical regression models prior to training or projection. These designate a set of control neurons and estimate their baseline activity within each sub-group. The subgroup may be, for example, each plate of cells, or each imaging day. The sub-groups are then aligned to the same level. Sub-groups may be estimated, for example, via best linear unbiased prediction (BLUP), which partially pools observed group-specific data with prior expectations generated via the entire data set. Importantly, aligning data in this way changes the value and interpretation of the fingerprints to reflect changes from baseline across a range of baselines, rather than the exact state of the neurons. This shift enables important applications for the autoencoder, such as the ability to derive fingerprints from novel cell types, which may have a different baseline. Thus, methods of the invention enable fingerprinting compound effects and disease phenotypes relative to a control for any disease.

Further, because some deep learning methods are prone to overfitting to the training data, in embodiments, methods of the invention may use a bootstrapping algorithm. Because some deep learning methods are prone to overfitting to the training data, in embodiments, methods of the invention use a bootstrapping algorithm to provide augmented training data, useful to avoid a machine learning system prone to overfitting. Prior art data augmentation methods have addressed overfitting by injecting noise into existing data or parameterizing the characteristics of the data set in order to generate similar synthetic data. In contrast, methods of the invention use bootstrapping to resample (e.g., with replacement) from within the training data to create augmented data without any requirement for synthetic data.

As noted above, methods of the invention collect data with single-neuron resolution. In embodiments, the hierarchical bootstrapping algorithm exploits this fact by resampling the neurons from the well with replacement to create another plausible example of the data that could have been collected from the well. Each measure is then aggregated at the well level using a measure-aware method, which applies the optimal aggregation strategy (mean, median, various degrees of trimmed mean) to each measure. These steps are repeated an arbitrary number of times for each well. In the data set described above, this resulted in a 100× increase in the size of the well-level training data. Importantly, this involves no synthetic data: all augmented samples are combinations of real data, maintaining all nonlinear dependencies between measures. To overcome memory constraints, this augmentation method may be applied in advance, during creation of the data stack, then saved to disk.

The bootstrapping algorithm resamples the data with replacement to create another plausible example of the data that could have been collected from the well. Each measure may be aggregated at the well level using a measure-aware method, which applies the optimal aggregation strategy (mean, median, various degrees of trimmed mean) to each measure. These steps are repeated an arbitrary number of times for each well. The analysis may provide e.g., a 100× increase in the size of the well-level training data. Importantly, this involves no synthetic data: all augmented samples are combinations of real data, maintaining all nonlinear dependencies between measures.

Methods and systems of the invention are useful for drug discovery. Methods include exposing electrically-excitable cells to a compound, measuring the electrical activity of the cells, identifying action potential features of the cells, and using a machine learning system to assess therapeutic efficacy of the compound based on the features identified. Importantly, the machine learning system is capable of producing a result regarding the therapeutic efficacy of a compound for any disease. This is accomplished by the nature of the machine learning system as described in the preferred embodiment above.

As noted above, the action potential features may be identified for a single cell and include one or more of spike rate, spike height, spike width, depth of afterhyperpolarization, timing of spike onset, timing of cessation of firing, an inter-spike interval of a first spike, extent of adaptation over a constant stimulation, a first derivative of spike waveform, and a second derivative of spike waveform. The machine learning system is trained to identify features of electrical activity associated with the therapeutic efficacy of a compound.

The features identified may be an output of tabular data with non-linear relationships between measures. Measured features may be stored numerically, e.g., as vectors, optionally scaled, e.g., to 0 to 1. Each feature (e.g., an optionally scaled numerical vector) may be input for a machine learning system such as an autoencoder neural network. An autoencoder is a type of artificial neural network used to learn efficient codings of unlabeled data and thus is an unsupervised learning technique. The autoencoder encodes the data to be usable by the machine learning system. As described above, the autoencoder may essentially be a representation-learning algorithm configured to map raw measurements onto a biological representation. The autoencoder may be trained with training data such as videos of cells of a known pathology or having a known gene target with samples of such cells both exposed to drugs and not (e.g., drugs of known effects and control samples). Known gene targets maybe genes with a disease-associated mutation. Methods of the invention develop a phenotype for all the diseases the machine learning system has been trained on, thus allowing for drug discovery for any disease.

The autoencoder may further be trained using hyperparameter tuning by optimizing the depth, width, nonlinearities, batch size, learning rate, momentum, gradient clipping, and training cycles of the autoencoder. These tuned hyperparameters have a large influence on model performance and utility.

The trained machine learning system is useful for detecting the effects of compounds. It is valuable to know how biological samples respond to a drug. For example, finding biologically active compounds in a screen, or finding the lowest dose with detectable activity. In pharmacology, biological activity or pharmacological activity describes the beneficial or adverse effects of a drug on living matter. This is difficult to do with high-dimensional readouts, because it is not known ahead of time which measurements will contain the differences, and the measurements themselves are not independent, a requirement for most common multiple comparisons procedures. Appropriate methods for such cases involve combined tests aggregated across features, and several computationally demanding nonparametric approaches including simulations and permutation methods.

To address this challenge, the invention provides a neuronal fingerprinting-based activity detector. In embodiments, the method calculates the fingerprints for each sample, then determines which fingerprints lie inside the “cloud of inertness” defined by the high-n replication of control wells. Samples that give a very low probability of being inert are then labeled as active. This technique is enabled by two assets: (1) a fitted fingerprinting algorithm, such as is described above, with which to find fingerprints and (2) control samples to populate the “cloud of inertness” at the center of the fingerprint space. The determination of the probability of inertness can be made using several computationally inexpensive techniques, including multivariate gaussian distributions and nonparametric kernel density estimation.

A machine learning system of the disclosure may be re-trained or updated. By retraining or updating the model, the model can become more specific and sensitive while reducing or eliminating issues such as intra-class correlation within a plurality of in vitro assays. As described above, an exemplary method of the invention includes removing action potential features from the training data, resampling from the training data, and re-training the machine learning system on the resampled data. Additional, un-analyzed action potential features may be added to the resampling data. Alternatively or additionally, the resampling data include duplicated action potential features. The model may be updated frequently using sentinel plates that provide standardized control signatures appropriate for the testing and tissue-culture conditions specific to that data set.

Additionally, methods are provided for finding the boundary specified by the union of the different techniques. The fingerprint is several orders of magnitude lower dimensionality than the raw data, making such approaches tractable. Fingerprint dimensions are significantly less correlated, with clear extensions to methods that encourage orthogonality, like Beta Variational Autoencoders, which enables the use of standard multiple comparisons corrections. The fingerprint dimensions are interpretable representations, allowing the activity detector to summarize the type and direction of activity.

In certain methods and systems of the disclosure a relational database is used. The database may include functional phenotypes derived from action potential features identified, for example, from cells expressing a particular neural disorder phenotype and/or caused by exposing cells to a therapeutic compound. The relational database may also include additional data attributable to the cells that exhibited the action potentials, such as cell type, neurological condition, mutations, and the like. In addition, the relational database may include data related to a particular known or putative therapeutic compound, such as structural features, active groups, concentration-dependent effects, known side effects, selectivity, potency, mechanisms of action, the ability to cross the blood-brain-barrier, cross reactivity with other compounds and the like.

The systems and methods of the present invention use optogenetics to create and record optical signals from changes in membrane potential caused when a cell exhibits an action potential. The time-varying signals produced by these optogenetic reporters are repeatedly measured (i.e., a movie is recorded) to chart the course of chemical or electronic states of living cells. The systems and methods of the invention can use a microscope to record time-varying signals (movies) produced by the optogenetic reporters of membrane potential as a video.

The present invention includes methods for reducing the size of this raw video data using a compression technique. The movie frames have high temporal correlation but are very noisy, so standard lossless compression or interframe difference lossless compression only achieve a maximal compression of ˜30%. Thus, the present invention provides methods and systems that reduce the size of the recorded data using a lossy compression method. Preferably, the lossy compression includes truncated principal component analysis (PCA), which discards noise but keeps almost all the information from the action potential signals.

PCA involves the calculation of a covariance matrix and its eigenvalue decomposition which scales quadratically with the number of pixels. A naïve implementation is therefore rather slow. A more computationally efficient algorithm uses block-wise processing. This is possible because signal correlations from stimulated cells are locally constrained. The present inventors have found that an aggressive compression of up to a factor of 200× can be achieved with minor loss of signal quality upon visual inspection.

To assure the critical information is maintained using this compression scheme, functional features can be generated using data that was compressed and using the data from before its compression. This can be used to assess possible signal degradation up to the end-point of phenotype discrimination for screening windows. The parcellation of the movie can be modified to use region-based tiling based on local intensity maxima of the mean movie frame. This allows for more efficient compression because pixels from single cells will tend to be contained within the same region.

Conservatively, the lossy compression methods describe herein reduce the data by a factor of less than about 200× without substantially compromising the downstream image segmentation or extracted voltage traces.

Advantageously, the lossy compression can act as a denoiser which can boost signal-to-noise ratios of the action potential signals.

Methods based on non-negative matrix factorization (NMF) can directly work with the compressed representation using truncated PCA. NMF-based methods are state-of-the-art algorithms developed for in vivo calcium imaging and have been modified to work with voltage-imaging. These methods solve a non-convex optimization problem. Thus, it is crucial to have good initial parameter conditions.

Database schema can also be used to provide more effective indexing through use of table partitioning and caching results that are queried often. For example, most queries against the database are for a specific project. A query of a table in a database may have to parse through properties for each spike waveform of every cell in the table. Partitioning this table decreases latency for most typical queries. Further, aggregated properties across spikes from the same cell are stored in a source-feature-table which can be precomputed, cached and retrieved without repeated computations.

Fluorescence values are extracted from raw movies by any suitable method. One method uses the maximum likelihood pixel weighting algorithm described in Kralj et al., 2012, Optical recording of action potentials in mammalian neurons using a microbial rhodopsin, Nat Methods 9:90-95. Briefly, the fluorescence at each pixel is correlated with the whole-field average fluorescence. Pixels that showed stronger correlation to the mean are preferentially weighted. This algorithm automatically finds the pixels carrying the most information, and de-emphasizes background pixels.

In movies containing multiple cells, fluorescence from each cell is extracted via methods known in the art such as Mukamel, 2009, Automated analysis of cellular signals from large-scale calcium imaging data, Neuron 63(6):747-760, or Maruyama, 2014, Detecting cells using non-negative matrix factorization on calcium imaging data, Neural Networks 55:11-19, both incorporated by reference. Those methods use the spatial and temporal correlation properties of action potential firing events to identify clusters of pixels whose intensities co-vary, and associate such clusters with individual cells.

Alternatively, a user defines a region comprising the cell body and adjacent neurites, and calculates fluorescence from the unweighted mean of pixel values within this region. In low-magnification images, direct averaging and the maximum likelihood pixel weighting approaches may be found to provide optimum signal-to-noise ratios. An image or movie may contain multiple cells in any given field of view, frame, or image. In images containing multiple neurons, the segmentation can be performed semi-automatically using an independent components analysis (ICA) based approach modified from that of Mukamel 2009. The ICA analysis can isolate the image signal of an individual cell from within an image.

The statistical technique of independent components analysis finds clusters of pixels whose intensity is correlated within a cluster, and maximally statistically independent between clusters. These clusters correspond to images of individual cells.

Spatial filters can be calculated to extract the fluorescence intensity time-traces for each cell. Filters are created by setting all pixel weights to zero, except for those in one of the image segments. These pixels are assigned the same weight they had in the original ICA spatial filter.

By applying the segmented spatial filters to the movie data, the ICA time course is broken into distinct contributions from each cell. Segmentation may reveal that the activities of the cells are strongly correlated, as expected for cells found together by ICA.

For individual cells, the sub-cellular details of action potential propagation can be represented by the timing at which an interpolated action potential crosses a threshold at each pixel in the image. Identifying the wavefront propagation may be aided by first processing the data to remove noise, normalize signals, improve SNR, other pre-processing steps, or combinations thereof. Action potential signals may first be processed by removing photobleaching, subtracting a median filtered trace, and isolating data above a noise threshold. The action potential wavefront may then be identified using an algorithm based on sub-Nyquist action potential timing such as an algorithm based on the interpolation approach of Foust, 2010, Action potentials initiate in the axon initial segment and propagate through axon collaterals reliably in cerebellar Purkinje neurons. J. Neurosci 30:6891-6902 and Popovic, 2011, The spatio-temporal characteristics of action potential initiation in layer 5 pyramidal neurons: a voltage imaging study, J Physiol 589:4167-4187, both incorporated by reference.

A sub-Nyquist action potential timing (SNAPT) algorithm highlights subcellular timing differences in action potential initiation. For example, the algorithm may be applied for neurons expressing a voltage reporter and a voltage actuator. Either the soma or a small dendritic region is stimulated via repeated pulses of blue light. The timing and location of the ensuing action potentials is monitored.

A first step in the temporal registration of spike movies may involve determining the spike times. Determination of spike times is performed iteratively. A simple threshold-and-maximum procedure is applied to the whole-field fluorescence trace, F(t), to determine approximate spike times, {T0}. Waveforms in a brief window bracketing each spike are averaged together to produce a preliminary spike kernel K0(t). A cross-correlation of K0(t) with the original intensity trace F(t) is calculated. Whereas the timing of maxima in F(t) is subject to errors from single-frame noise, the peaks in the cross-correlation, located at times {T}, are a robust measure of spike timing. A movie showing the mean action potential propagation may be constructed by averaging movies in brief windows bracketing spike times {T}. Typically, 100-300 action potentials are included in this average. The action potential movie has high signal-to-noise ratio. A reference movie of an action potential is thus created by averaging the temporally registered movies (e.g., hundreds of movies) of single action potentials.

Spatial and temporal linear filters may further decrease the noise in an action potential movie. A spatial filter may include convolution with a Gaussian kernel, typically with a standard deviation of 1 pixel. A temporal filter may be based upon Principal Components Analysis (PCA) of the set of single-pixel time traces. The time trace at each pixel is expressed in the basis of PCA eigenvectors. Typically, the first 5 eigenvectors are sufficient to account for >99% of the pixel-to-pixel variability in action potential waveforms, and thus the PCA eigen-decomposition is truncated after 5 terms. The remaining eigenvectors represented uncorrelated shot noise.

The eigenvectors resulting from a principal component analysis (PCA) can be used in a smoothing operation to address noise. Photobleaching or other such non-specific background fluorescence may be addressed by these means.

A smoothly varying spline function may be interpolated between the discretely sampled fluorescence measurements at each pixel in this smoothed reference action potential movie. The timing at each pixel with which the interpolated action potential crosses a user-selected threshold may be inferred with sub-exposure precision. The user sets a threshold depolarization to track (represented as a fraction of the maximum fluorescence transient), and a sign for dV/dt (indicating rising or falling edge). The filtered data is fit with a quadratic spline interpolation and the time of threshold crossing is calculated for each pixel.

The timing map may be converted into a high temporal resolution SNAPT movie by highlighting each pixel in a Gaussian time course centered on the local action potential timing. The SNAPT fits are converted into movies showing action potential propagation as follows. Each pixel is kept dark except for a brief flash timed to coincide with the timing of the user-selected action potential feature at that pixel. The flash followed a Gaussian time-course, with amplitude equal to the local action potential amplitude, and duration equal to the cell-average time resolution, σ. Frame times in the SNAPT movies are selected to be ˜2-fold shorter than σ. Converting the timing map into a SNAPT movie is for visualization; propagation information is in the timing map.

Environmentally sensitive fluorescent reporters for use with the present invention include rhodopsin-type transmembrane proteins that generate an optical signal in response to changes in membrane potential, thereby functioning as optical reporters of membrane potential. Archaerhodopsin-based protein QuasAr2 and QuasAr3, are excited by red light and produce a signal that varies in intensity as a function of cellular membrane potential. These proteins can be introduced into cells using genetic engineering techniques such as transfection or electroporation, facilitating optical measurements of membrane potential. The invention can also be used with voltage-indicating proteins such as those disclosed in U.S. Patent Publication 2014/0295413, the entire contents of which are incorporated herein by reference.

In addition to fluorescent indicators, light-sensitive compounds have been developed to chemically or electrically perturb cells. Using light-controlled activators, stimulus can be applied to entire samples, selected regions, or individual cells by varying the illumination pattern. One example of a light-controlled activator is the channelrhodopsin protein CheRiff, which produces a transmembrane current of increasing magnitude roughly in proportion to the intensity of blue light falling on it. In one study, CheRiff generated a current of about 1 nA in whole cells expressing the protein when illuminated by about 22 mW/cm2 of blue light.

The systems and methods of the invention may also use additional reporters and associated systems for actuating them. For example, proteins that report changes in intracellular calcium levels may be used, such as a genetically-encoded calcium indicator (GECI). The plate reader may provide stimulation light for a GECI, such as yellow light for RCaMP. Exemplary GECIs include GCaMP or RCaMP variants such for example, jRCaMP1a, jRGECO1a, or RCaMP2. In one embodiment, the actuator is activated by blue light, a Ca2+ reporter is excited by yellow light and emits orange light, and a voltage reporter is excited by red light and emits near infrared light.

Optically modulated activators can be combined with fluorescent indicators to enable all-optical characterization of specific cell traits such as excitability. For example, the Optopatch method combines an electrical activator protein such as CheRiff with a fluorescent indicator such as QuasAr2. The activator and indicator proteins respond to different wavelengths of light, allowing membrane potential to be measured at the same time cells are excited over a range of photocurrent magnitudes. Optopatch includes the contents of U.S. Pat. Nos. 10,613,079 and 9,594,075, both incorporated by reference for all purposes.

All-optical measurements provide an attractive alternative to conventional methods like patch clamping because they do not require precise micromechanical manipulations or direct contact with cells in the sample. Optical methods are much more amenable to high-throughput applications. The dramatic increases in throughput afforded by all-optical measurements have the potential to revolutionize study, diagnosis, and treatment of these conditions.

Methods and systems of the disclosure may use a multi-well plate microscope to record action potentials of cells in wells of the plate. For example, methods and systems of the invention may employ a multi-well plate microscope for illuminating a sample with near-TIR light in a configuration that allows living cells to be observed and imaged within wells of a plate. The microscope illuminates the sample from the side rather than through the objective lens, which allows more intense illumination, and a corresponding lower numerical aperture and larger field of view. By using illumination light at a wavelength distinct from the wavelength of fluorescence, the TIR microscope allows the illumination wavelengths to be nearly completely removed from the image with optical filters, resulting in images that have a dark background with bright areas of interest. The microscope can observe fluorescence to provide indicative measures of cellular action potentials from which action potential features/parameters are extracted.

Fluorescent reporters of membrane action potential, such as QuasAr2 and QuasAr3, require intense excitation light in order to fluoresce. Low quantum efficiency and rapid dynamics demand intense light to measure electrical potentials. The illumination subsystem is therefore configured to emit light at high wattage or high intensity. Characteristics of a fluorophore such as quantum efficiency and peak excitation wavelength change in response to their environment. The intense illumination allows that to be detected. Autofluorescence caused by the intense light is minimized by the microscope in multiple ways. The use of near-TIR illumination exposes only a bottom portion of each well to the illumination light, thereby reducing excitation of the culture medium or other components of the device. Additionally, the microscope is configured to provide illumination light that is distinct from imaging light. Optical filters in the imaging subsystem filter out illumination light, removing unwanted fluorescence from the image. Cyclic olefin copolymer (COC) dishes for culturing cells enable reduced background autofluorescence compared to glass. The prism is coupled to the multi-well plate through an index-matching low-autofluorescence oil. The prism is also composed of low autofluorescence fused silica.

The microscope is configured to optically characterize the dynamic properties of cells. The microscope realizes the full potential of all-optical characterization by simultaneously achieving: (1) a large field of view (FOV) to allow measurement of interactions between cells in a network or to measure many cells concurrently for high throughput; (2) high spatial resolution to detect the morphologies of individual cells in wells and facilitate selectivity in signal processing; (3) high temporal resolution to distinguish individual action potentials; and (4) a high signal-to-noise ratio to facilitate accurate data analysis. The microscope can provide a field of view sufficient to capture tens or hundreds of cells. The microscope and associated computer system provide an image acquisition rate on the order of at least 1 kilohertz, which corresponds to a very short exposure time on the order of 1 millisecond, thereby making it possible to record the rapid changes that occur in electrically active cells such as neurons. The microscope can therefore acquire fluorescent images using the recited optics over a substantially shorter time period than prior art microscopes.

The microscope achieves all of those demanding requirements to facilitate optically characterizing the dynamic properties of cells. The microscope provides a large FOV with sufficient resolution and light gathering capacity with a low numerical aperture (NA) objective lens. The microscope can image with magnification in the range of 2× to 6× with high-speed detectors such as sCMOS cameras. To achieve fast imaging rates, the microscope uses extremely intense illumination, typically with fluence greater than, e.g., 50 W/cm2 at a wavelength of about 635 nm up to about 2,000 W/cm2.

Despite the high power levels, the microscope nevertheless avoids exciting nonspecific background fluorescence in the sample, the cell growth medium, the index matching fluid, and the sample container. Near-TIR illumination limits the autofluorescence of unwanted areas of the sample and sample medium. Optical filters in the imaging subsystem prevent unwanted light from reaching the image sensor. Additionally, the microscope prevents unwanted autofluorescence of the glass elements in the objective lens by illuminating the sample from the side, rather than passing the illumination light through the objective unit. The objective lens of the microscope may be physically large, having a front aperture of at least 50 mm and a length of at least 100 mm, and containing numerous glass elements.

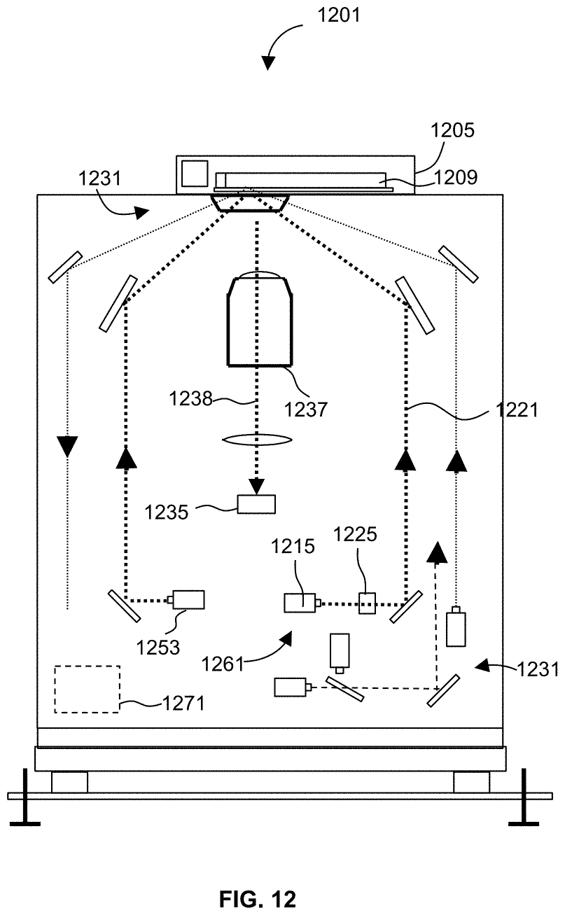

FIG. 12 shows components of an exemplary microscope 1201. The microscope includes a stage 1205 configured to hold a multi-well plate 1209; an excitation light source 1215 for emitting a beam of light mounted within the microscope; and an optical system 1261 that directs the beam towards the stage from beneath. The optical system comprises a homogenizer 1225 for spatially homogenizing the beam. The microscope 1201 includes or is communicatively coupled to a computer 1271 or computing system hardware for performing or controlling various functions.

The computer 1271 may include a machine learning system to identify action potential features and/or generate a functional phenotype.

The microscope 1201 may include a light patterning system 1231. The stage 1205 is preferably a motorized x,y translational stage.

The microscope 1201 includes an image sensor 1235. The image sensor may be provided as a digital camera unit such as the ORCA-Fusion BT digital CMOS camera sold under part #C15440-20UP by Hamamatsu Photonics K.K. (Shizuoka, JP) or the ORCA-Lightning digital CMOS camera sold under part #C14120-20P by Hamamatsu Photonics K.K. Another suitable camera to use for sensor 1235 is the back-illuminated sCMOS camera sold under the trademark KINETIX by Teledyne Photometrics (Tucson, Ariz.).

The microscope may also include an imaging lens 1237 such as a suitable tube lens. The lens 1237 may be an 85 mm tube lens such as the ZEISS Milvus 85 mm lens. With such imaging hardware, the microscope can image an area with a diameter of 5.5 mm in a 96-well plate and the full 3.45 mm well width of a 384-well plate.

The microscope 1201 preferably includes a control system comprising memory connected to a processor operable to move the translational stage to position individual wells of the multi-well plate in the path of the beam. Optionally, the microscope 1201 includes an excitation light source 1215 mounted within the microscope for emitting a beam 1221 of light. The optical system 1261 directs the beam 1221 towards the stage from beneath.

The microscope 1201 may optionally include a secondary light source 1253. The secondary light source 1253 may have its own optical system that share some similarities with the optical system 1261. However, including the optical system 1261 and the secondary light source 1253 with its own optical system allows those systems to be operated independently, simultaneously or not. In some embodiments, the secondary light system is operated a different (e.g., much higher) power than the optical system 1261. The secondary light source 1253 and its system may be used for calibration or to address optogenetic proteins that operate best at a different power than sets of optogenetic proteins addressed by the optical system 1261.

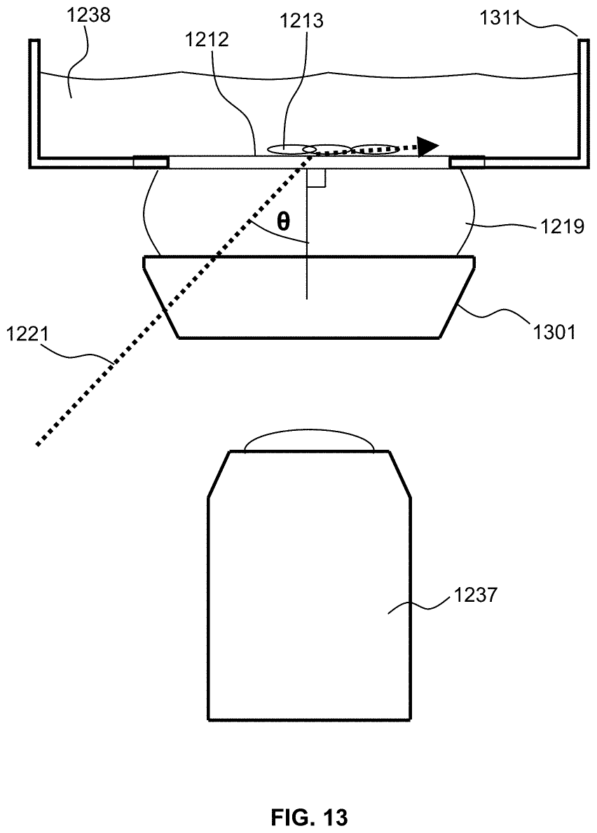

FIG. 13 shows a prism 1301 that guides the beam 1221 towards the sample 1213. The optical system 1261 includes a prism 1301 immediately beneath the stage, whereby the beam enters a side of the prism and passes into a well 1311 of the plate. As shown, an aqueous sample 1238 includes living cells 1213 on a bottom surface 1212 of a well 1311. Optionally, index-matched lens oil 1219 optical couples the prism 1301 to the bottom 1212 of the well. Preferably, when a well 1311 of the plate containing an aqueous sample 1238 is positioned above the prism 1301, the prism directs the beam 1221 into the sample at angle theta that avoids total internal reflection within the bottom 1212 of the well of the plate. As shown, when a well of the plate containing an aqueous sample is positioned above the prism, the prism directs the beam into the aqueous sample at an angle of refraction that restricts light to about the bottom ten (optionally twenty) microns of the well.

The microscope, described herein, which can be used with the systems and methods of the disclosure can include all of its optical components positioned underneath a well of a multi-well plate such that illumination occurs from the side rather than through the objective lens. The side illumination allows the microscope to have more intense illumination and a larger field of view.

Optionally, an area above the stage is unencumbered by optical elements such as prisms. That configuration allows for physical access to the sample and control over its environment. Thus, the sample can be, for example, living cells in a nutrient medium. That configuration solves many of the problems associated with traditional TIRF microscopes. In particular, a thin region of sample cells can be illuminated with a near-TIR beam without having to physically interfere with the cells by loading them into a flow chamber. Instead, living cells in an aqueous medium such as a maintenance broth can be observed. The sample can be further analyzed from above with electrodes or other equipment as desired. The microscope can be used to image cells expressing fluorescent voltage indicators. Since the components do not interfere with the sample, living cells can be studied using a microscope of the invention. Where a sample includes electrically active cells expressing fluorescent voltage indicators, the microscope can be used to view voltage changes in, and thus the electrical activity of, those cells to derive action potential features.

Moreover, the microscope includes systems for spatially-patterned illumination, useful to selectively illuminate only specific cells within a sample.

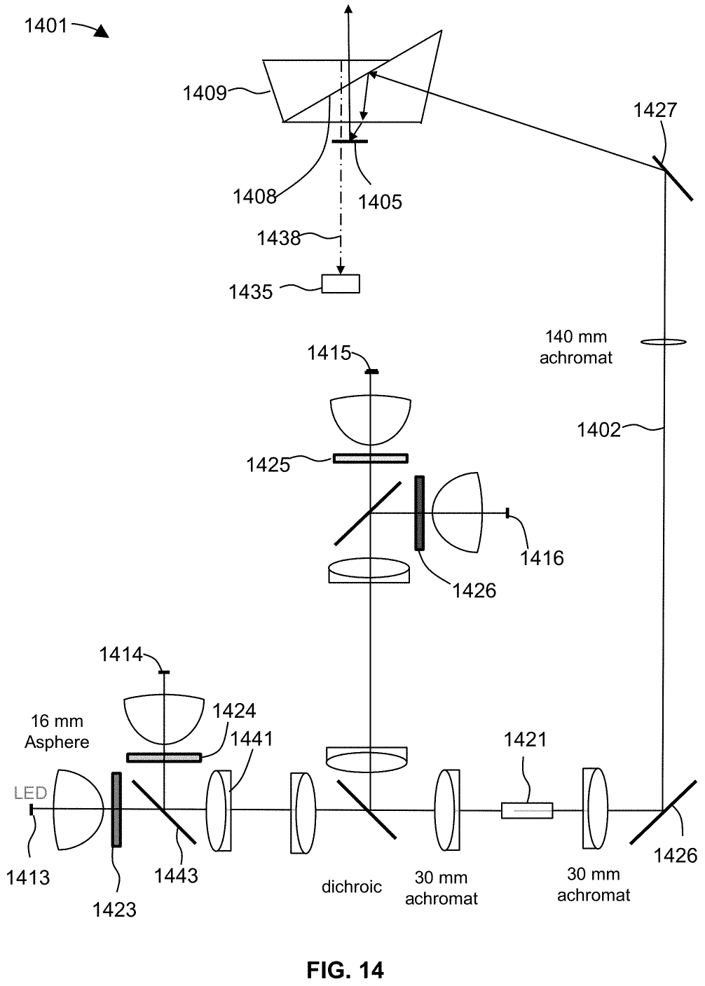

FIG. 14 shows an optical light patterning system 1401 to spatially pattern light of multiple wavelengths onto a sample. The light patterning system 1401 includes a first light source 1413 for emitting a beam 1402 of light. The beam of light reflects from a digital micromirror device (DMD) 1405. The DMD 1405 forms the beam 1402 into a pattern. The patterned beam is imaged onto the sample. The DMD will enable fully synchronized 100 μs pattern refresh for fast single-cell stimulation to measure individual synaptic connections or slightly delayed pulses on connected neurons to probe spike-timing dependent plasticity. The light patterning system may optionally include a second light source 1414. The first light source preferably sends light of a first wavelength into the beam 1402. This may be done using a filter 1423 for the first wavelength.

A dichroic mirror 1443 may selectively reflect light of a second wavelength from the second light source 1414 into the beam 1402. The light patterning system 1401 may include one or any number of lens element(s) 1441, such as 30 mm achromatic doublets, to guide light onto any dichroic mirror(s) 1443 or to collimate the beam 1402. The second light source 1414 may provide light at the second wavelength using a second filter 1424 specific for the second wavelength. The light patterning system 1401 may include a third light source 1415, a third filter 1425, and optionally a fourth light source 1416 and a fourth filter 1426. In preferred embodiments, once light from various wavelengths is joined in the beam 1402 the beam 1402 is passed through a light pipe 1421.

One optional embodiment uses four light sources with four wavelengths: UV (380 nm), blue (470 nm), yellow/green (560 nm), and red (625 nm). The UV (380 nm) may be useful for imaging EBFP2 or mTagBFP2 imaging or intracellular calcium. A power of 50 mW/cm2 may be sufficient. The blue (470 nm) may be used to image CheRiff (e.g., at 250 to 500 mW/cm2 to open >95% of channels), Chronos (e.g., at 500 mW/cm2 to open a majority of channels), FLASH, or other such proteins. The yellow/green (560 nm) may be used to image jRGECO1a (80 mW/cm2 at 560 nm for neurons, or 25 mW/cm2 for cardiomyocytes), VARNAM, or other proteins. The red (625 nm) may be useful for measuring target proteins with Alexa647 (e.g., at 50 mW/cm2), or cellular activity with BeRST (e.g., 1-20 W/cm2 for neurons).

The light patterning system 1401 may include one or any number of round mirrors 1426 to guide the beam 1402 from the light source 1413 (typically mounted to a solid frame or board) to the sample. The light patterning system 1401 includes an adjustable round mirror 1427 that controls the final angle by which light approaches the prism assembly 1409. In a preferred embodiment, the light pattern system 1401 includes a prism assembly 1409 that includes one or more prisms to guide the light onto the DMD 1405 and on to the sample. The prisms may preferably have a refractive index that matches a refractive index of a material that forms a bottom of a multi-well plate. For example, the microscope 1201 may be designed for use with a plate such as the glass bottom microplates with 24, 96, 384, or 1536 wells sold under the trademark SENSOPLATE by MilliporeSigma (St. Louis, Mo.). Such microplates have dimensions that include 127.76 mm length and 85.48 mm width. The microplates include borosilicate glass (175 μm thick).

The prism assembly 1408 may include a dichroic mirror 1408 that bounces select wavelengths of light off of the DMD 1005 and permits other select wavelengths to pass through at a near-TIR angle to thereby illuminate the sample over just the bottom 10 to 20 microns of the well. Here, near-TIR can be understood to mean that the angle is less than the critical angle by which the light coming from the side will exhibit total internal reflection in part of the multi-well plate hardware (e.g., will NOT exhibit TIR in the borosilicate glass bottom of the plate) but is nevertheless quite close to that, e.g., preferably within 10 degrees of the critical angle, more preferably within 5 degrees of the critical angle for TIR, most preferably within 2 degrees of the critical angle.

As shown, a sample that is imaged emits light 1438 that passes towards an imaging sensor 1435 (e.g., through a tube lens, not pictured). Because of the dichroic mirror, the sample can be illuminated with spatially pattern light, also illuminated from the side by near-TIR light that pass through only about the bottom 10 microns of the sample well (both from beam 1002), and also emit emitted light 1438 that is captured by the sensor 1435 to record a movie.

Any suitable digital light processor or spatial patterning mechanism may be used as the DMD 1405. In some embodiments, the DMD 1405 is a Vialux V9601-VIS DMD system with a 1920×1200 pixel array of micromirrors at an 10.8 μm pitch and a 20.7×13 mm array size. The light patterning system may optionally include a tube lens, such as a Zeiss Milvus 135 mm, to provide (e.g., 2.7×) demagnification onto the sample.

In the depicted embodiment, each light source 1413 is a 3×3 mm Luminus LED imaged onto 6×6 mm light pipe 1421 maintaining source etendue. The 4-lens design (2 4-f imaging systems) from LED to light pipe increases light collection efficiency and minimizes angular content. The depicted light patterning system 1401 includes at least three (e.g., four) light sources 1413, 1414, 1415, 1416 for emitting at least three beams at three distinct wavelengths. Preferably the light patterning system 1401 has one or more dichroic mirrors 1443 to join the three beams in space and pass the three beams through a homogenizer and/or the light pipe 1421. The light pipe 1421 homogenizes the source and ensures good overlap of four LED colors. Light from the light pipe 1421 is passed along towards the DMD.

The microscope 1201 may include an excitation light source 1215 mounted within the microscope for emitting a beam 1221 of light. The optical system 1261 directs the beam 1221 towards the stage at an angle from beneath. One potential issue is aberration that could affect a shape of the beam 1221. Thus, preferably, the microscope 1201 avoids non-uniform illumination of the cells 1213 by including, in the optical system 1261, a homogenizer 1225 for spatially homogenizing the beam 1221. Different methods of laser beam homogenization may be used to create a uniform beam profile. For example, homogenization may use a lens array optic or a light pipe rod.

An exemplary method for imaging samples using the microscope, as described herein, includes positioning a multi-well plate on the microscope stage, the plate having at least one cell living on a bottom surface of a well. Imaging is performed to obtain an image of the cell. The image is processed to “mask” the surface on the bottom of the well, i.e., to create a spatial mask identifying areas of the bottom surface occupied by the cell and areas not occupied by the cell. Using the mask, the computer signals the DMD to selectively activate micromirrors of the DMD that subtend the cell using the spatial mask. Then, using the light source, the microscope illuminates the sample by shining light onto the DMD to thereby specifically reflect light onto the areas of the bottom surface occupied by the cell while not reflecting any of the light onto the areas not occupied by the cell.

The method may include creating a spatial mask for cells in each of a plurality of wells of the multi-well plate; holding the spatial masks in memory; and using the spatial masks and DMD to selectively illuminate the cells in the plurality of wells in a serial manner. Optionally, the DMD is controlled by a computer comprising a process coupled to a non-transitory memory system, the memory system having the spatial masks stored therein.

For robust high-throughput operation, the systems and methods of the disclosure may employ software tools e.g., automation and control software use with the microscope to, for example, apply optogenetic stimuli, (e.g., a blue-light stimuli), record high-speed video data, move between wells and operate a pipetting robot for automated compound addition. Tools may include analysis software to extract voltage vs. time traces from each neuron in each multi-GigaByte video. The reduced data includes fluorescence traces proportional to transmembrane voltage, identified action potentials and extracted action potential features/parameters, as well as associated metadata such as cell type, compound, and compound concentration, which may be stored in a relational database.

### EXAMPLES

### Example 1: Automated Action Potential Feature Extraction Using hiPSC Expressing Optogenetic Proteins

Human induced pluripotent stem cells (hiPSC) were differentiated into hiPSC-derived motor neurons. The cells expressed an optogenetic proteins from the Optopatch toolkit (optical stimulation plus optical voltage reporting, e.g., CheRiff & QuasAr), which allows simultaneous optical stimulation and recording of neuronal action potentials.

The channelrhodopsin CheRiff enables action potential stimulation with blue light and the voltage-sensitive fluorescent protein QuasAr enables high-speed electrical recordings with red light. A microscope, as disclosed herein, obtained simultaneous voltage recordings from >100 individual neurons over a large (0.5×4 mm) field of view (FOV) with 1 ms temporal resolution and high signal-to-noise ratio (SNR). A digital micromirror device (DMD) in the microscope projected a fully reconfigurable optical pattern to sequentially stimulate cells while recording from many post-synaptic partners. A computer system provided fully automated analyses to identify each individual neuron and calculate its voltage trace.

In every trace the spikes were detected and the key spike shape and timing parameters were computed. Since each cell fired many action potentials, a wealth of information could be extracted to, for example, distinguish cell type, cell state, disease phenotype and pharmacological response. Additionally, the electrode-free recordings minimally perturbed the cells, enabling the recording of the same neurons before and after compound addition, which allowed identification of compound effects on different neuronal sub-types, which overcomes the biological “noise” of highly heterogeneous neuronal responses. In addition to cell autonomous excitability and firing patterns, the system makes it possible to study synaptic transmission, long term potentiation/depression and network and circuit behavior.

The hiPSC-derived motor neurons were put into wells of a multi-well plate and interrogated with a stimulus protocol (blue light pulses) designed to probe a broad range of spiking behaviors using a microscope as described herein. Recordings of the fluorescent signals in response to the stimulus were taken by the microscope.

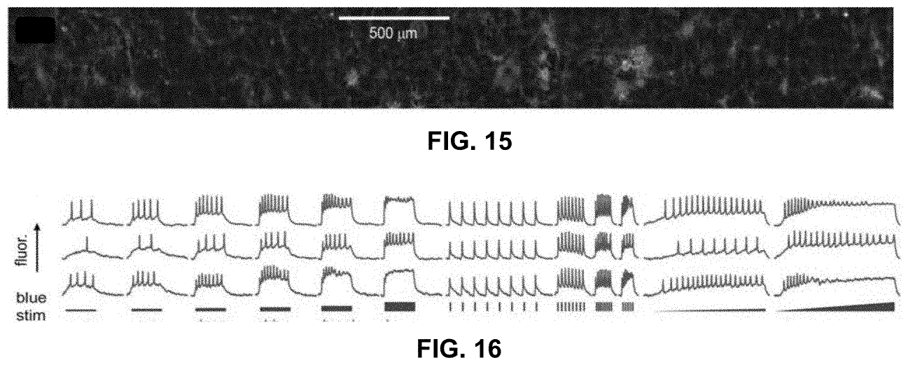

FIG. 15 shows an image from the recording with overlay (colored regions) of hiPSC-derived motor neurons which were identified by automated analysis using the system.

FIG. 16 shows voltage recordings from hiPSC-derived motor neurons identified by the automated analysis using a machine learning system. Voltage recordings from selected cells, and the blue stimulus used to evoke firing: steps, pulse trains, and ramps are shown.

Pixels in the recording that captured fluorescence from the reporters of membrane potential in each neuron co-varied in time following that cell's unique firing pattern. A temporal covariance was used to generate a weight mask for each cell (colored regions in FIG. 15). Masked pixels were averaged for each frame in the recording to calculate the traces. Each FOV was recorded twice, before and after addition of potassium channel opener ML-213.

The traces in FIG. 16 demonstrate the underlying variability in neuronal behavior. Recordings from many neurons were averaged to capture the effect the compound had on the action potentials of the neurons. From the traces, each individual, recorded action potential was identified.

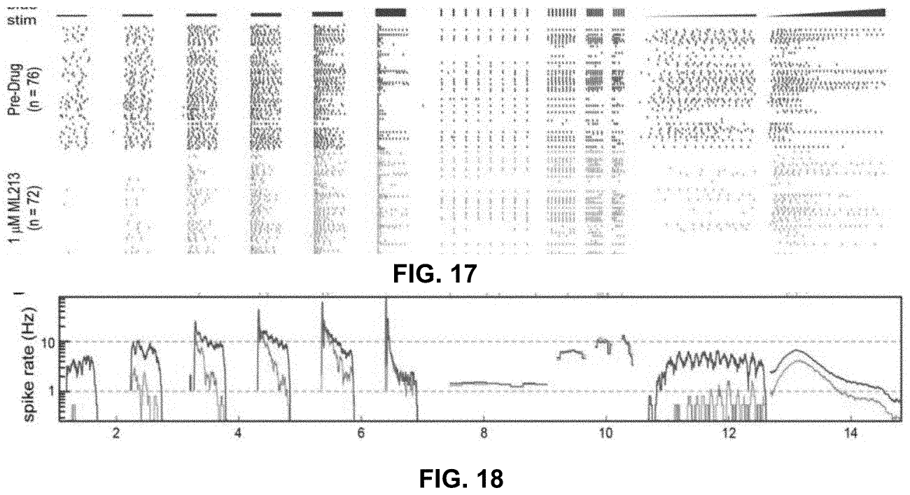

FIG. 17 provides a raster plot where each point is an identified action potential and each row is a neuron from a single field of view. The dark-colored plot was derived from recordings of the neurons prior to the addition of ML-213, a potassium channel blocker that lowers resting potential and suppresses action potential firing in the neurons. The light-colored plot was derived from recordings after the addition of 1 μM of ML-213.

FIG. 18 provides the spike rate averaged over the cells (the firing rate).

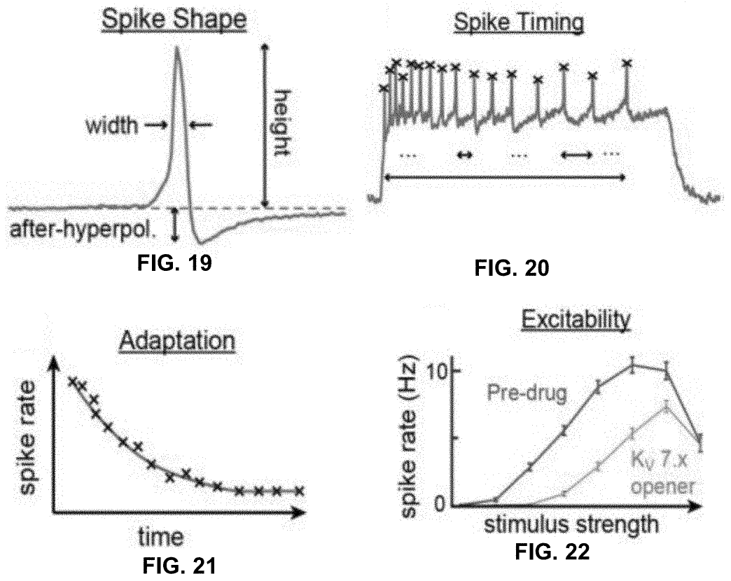

FIG. 19 provides spike shape parameters extracted from the action potentials.

FIG. 20 provides spike timing parameters extracted from the action potentials.

FIG. 21 provides the adaptation average over the cells as extracted from the action potentials.

The spike shape, spike timing properties, and adaptation were automatically extracted using a machine learning system for each cell and measured as a function of the stimulus.

FIG. 22 shows the clear reduction in neuronal excitability caused by ML-213. All parameters were automatically extracted by the parallelized analysis in the cloud, stored in the database, and figures are automatically generated by the system. The stimulus-dependent extracted values, greatly reduced in number and complexity from the raw video data, show that action potential features as described herein can serve as the substrate for more detailed analysis for distinguishing cell type, cell state, disease phenotype and pharmacological response. Further, to provide an analysis of greater depth and breadth, approximately 300 parameters could be extracted from the action potentials of each cell. A machine learning system can identify a key subset of these features to generate a functional phenotype.

### Example 2: Compound Screening Using Action Potential Features from hiPSC Expressing Optogenetic Proteins

In this example, iPSC-derived excitatory cortical neurons (NGN2) were grown for 30 days in a culture. The neurons expressed Optopatch proteins as described in Example 1. Two sets of neurons were grown. The first was a wildtype control line. The second had a confidential loss of function mutation caused by a knockout (KO) of a gene to model a neural disease.

The cells were stimulated using blue light as described in Example 1 and their action potentials recorded as voltage traces. Recordings were made of the control cells and disease-model cells when stimulated in the absence of any test compound. Recordings were also made of the disease-model cells when stimulated in the presence of the promiscuous potassium channel blocker 4-AP and the promiscuous sodium channel blocker lamotrigine.

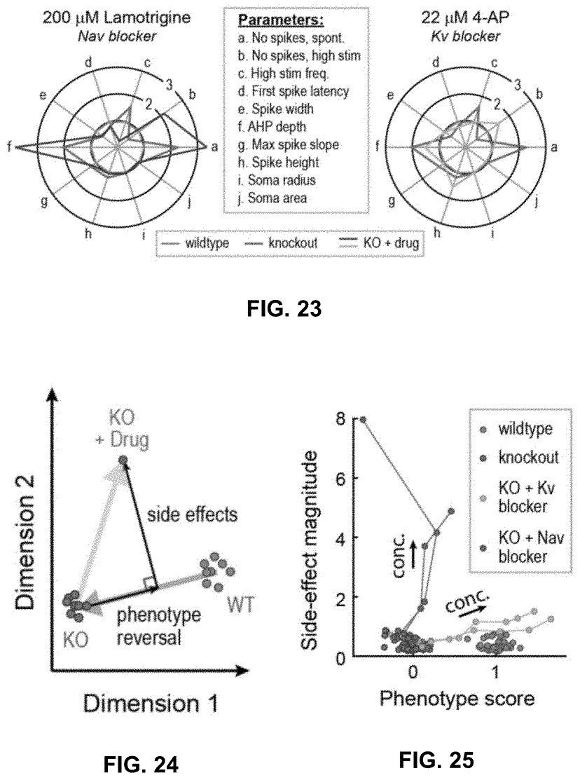

FIG. 23 provides radar plots representing functional phenotypes generated by a machine learning system using action potential features extracted from the recorded action potentials when the cells were stimulated by blue light. The values for the features are normalized to the control cell recordings. The left plot shows a function phenotype of extracted features from the disease-model cells in the presence of the sodium channel blocker. The right shows the phenotype from the disease-model cells in the presence of the potassium channel blocker. The differences in the recorded traces, select features of which are provided on the radar plots, show the functional phenotype of the disease-model. 4-AP substantially reversed the phenotype, as shown in the radar plot by bringing the action potential features of the disease-model cells closer to that of the control cells when compared to the disease-model cells in the absence of 4-AP. In contrast, lamotrigine perturbed behavior but did not reverse the phenotype.

The radar plots allow easy visualization of disease phenotype and compound effects.

FIG. 24 is a diagram illustrating phenotype reversal and “side effects” described by mapping extracted action potential features on the ˜300-dimensional space of recorded parameters, only two of which are shown. Extracted features for the control cell (WT) wells (green) are clustered as are those for the KO cells (red). The vector between these populations represents the phenotype (red). Drug effects (blue) are deconstructed into components along (phenotype reversal) and orthogonal to (side effects) the phenotype vector. An ideal drug would undo the effects of the mutation and move the well from the KO cluster to the control cell cluster.

FIG. 25 is a plot showing many wells projected onto the phenotype/side effect space. WT and KO wells are well separated along the phenotype direction. Application of the two compounds (8 concentrations from 0.28 to 600 μM) from FIG. 25 have increasing effects on KO cell behavior as the concentration increases. 4-AP moves cell behavior toward and beyond WT behavior, while lamotrigine moves behavior away from both WT and KO. The connected drug points are in order of increasing concentration, and the two lines are experimental replicates on two consecutive weeks of experiment.

Thus, this example shows that action potential features can be used to accurately ascertain cellular response to drug compounds, including at varied concentrations.

### Example 3: Characterizing the Effects on Action Potential Features Caused by a Number of Compounds

This example shows that the presently disclosed systems and methods can be used to derive functional phenotypes characterizing the changed behavior of cells in response to a number of different compounds that effect varied targets.

E18 rat hippocampal neurons were cultured for 14 days and caused to express Optopatch proteins as described in Example 1. The cells were stimulated in the presence of XE-991 (a Kv7.x blocker), ML-213 (a Kv7.x opener), a-Dendrotoxin (a Kv1.x blocker), OXO-M (a muscarinic agonist), 4AP (a promiscuous Kv blocker), Isradipine (a Cav1.x blocker), or a control vehicle.

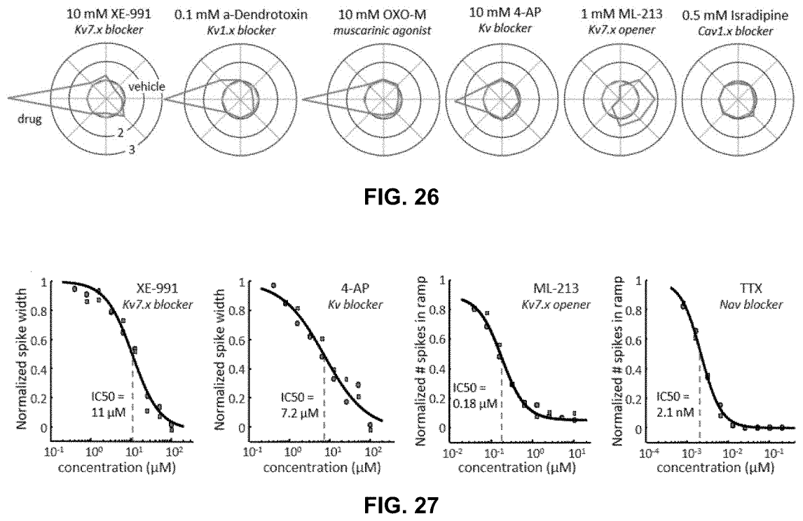

FIG. 26 provides radar plots representing generated functional phenotypes indicative of the drug-induced changes in neuronal spiking behavior along many dimensions. The action potential feature values were normalized to those for the cells simulated in the presence of the control vehicle. As shown in the radar plots, each compound provided a discernable and unique functional phenotype. For example, XE-991, a voltage-gated potassium channel Kv7.x blocker, and ML-213, a Kv7.x opener, drove cellular response, as expected.

FIG. 27 provides concentration response curves for the cells in the presence of varied concentrations of the compounds. Each symbol represents >100 cells in one well and all measurements were obtained in a single day. Thus, the present systems and methods can not only elucidate therapeutic responses of various compounds, but also show concentration-dependent responses. Moreover, as the measurements were taken in a single day, the presently disclosed systems and methods enable fast, high-throughput drug screening.

### Example 4: Consistent and Repeatable Measurements of Pharmacological Effects and Disease Phenotypes

This example shows that the measurements obtained using the systems and methods of the disclosure are uniform, consistent, and repeatable.

E18 rat hippocampal neurons were cultured for 14 days and caused to express Optopatch proteins as described in Example 1. The cells were placed in wells of a 96-well plate. ML-213 at 1 μM was added to alternating columns of the plate and a control vehicle added to the remaining columns. The cells in all wells were stimulated and their action potentials recorded using a microscope as described in Example 1.

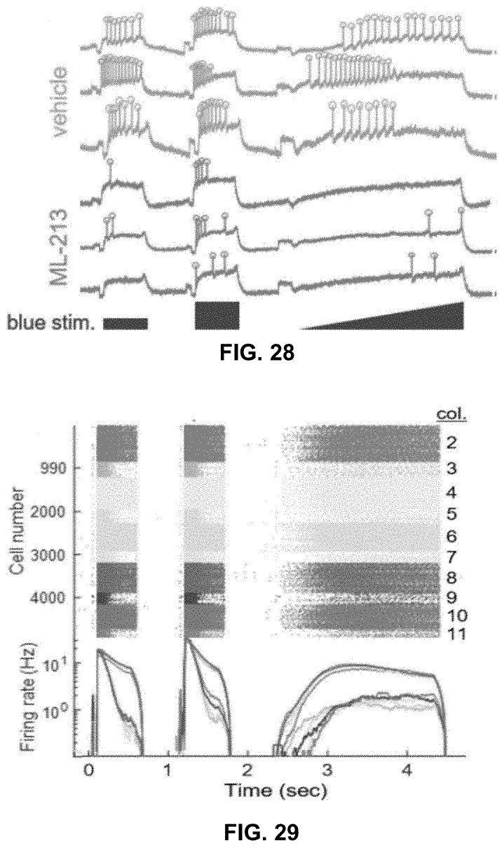

FIG. 28 shows high SNR fluorescent voltage recordings obtained from the microscope of the neurons in the 96-well plate. The blue light stimulus is shown below.

FIG. 29 shows a raster plot showing spikes recorded in each column.

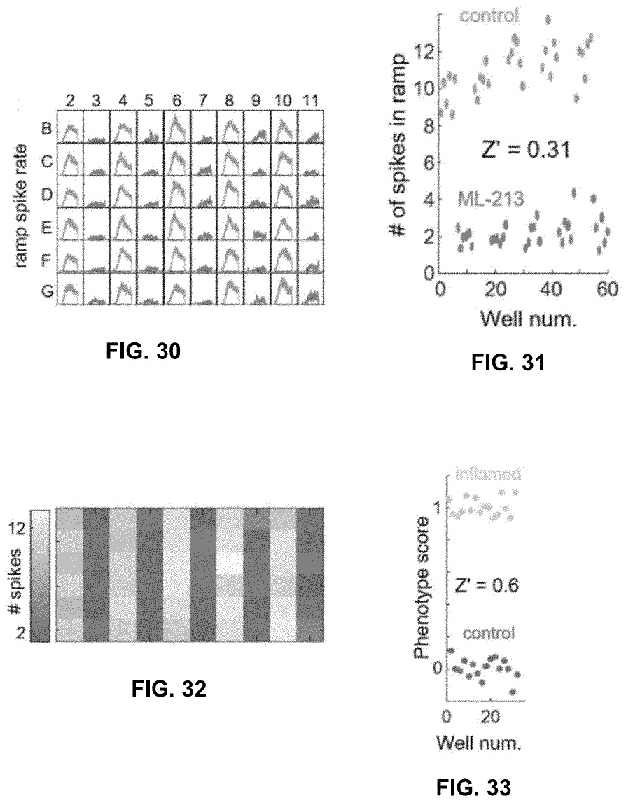

FIG. 30 provides the average firing rate during the blue light stimulus ramp for each well. As shown, ML-213 dramatically reduces firing rate as the vehicle wells (green) and ML-213 wells (red) are clearly distinguishable.

FIG. 31 provides a heat map showing the number of spikes recorded in each well during the blue light stimulus ramp.

FIG. 32 provides a plot of the average number of spikes recorded for individual cells in each well during the blue light stimulus ramp. The calculated Z′ of 0.31 indicates a failed call in 1 of 73,000 wells, and shows that measurements are consistent across wells, allowing the systems and methods of the invention to be used in drug discovery screens.

FIG. 33 provides a plot of the average number of spikes recorded for individual DRG neurons in wells of a 96-well plate. Each column of the plate was contacted with either a control vehicle or a cocktail of inflammatory mediators found in joints of arthritis patients. As expected, the cells in wells with the mediators fired more action potentials than did those in wells with the control vehicle. The Z′ score again indicates the repeatability and consistency of the presently disclosed systems and methods to accurately distinguish phenotypes of cells in the presence of different biological conditions and/or the presence of different drug compounds. Inflammatory mediator cocktails may be compositions as described in WO 2018/165577, incorporated herein by reference.

### Example 5: Action Potential Feature Extraction Using Isogenic Disease Models

The presently disclosed systems and methods can be applied to many neuronal types and/or disease models to produce functional phenotypes.

Wildtype cells were obtained and a CRISPR/Cas9 system was used to knockout a gene to produce isogenic clones that were expanded, converted to neurons, and caused to express Optopatch proteins as described in Example 1. The knockout caused the neurons to exhibit a monogenic epilepsy phenotype due to a loss of function. The knockout created either heterozygous or homozygous for the loss of function.

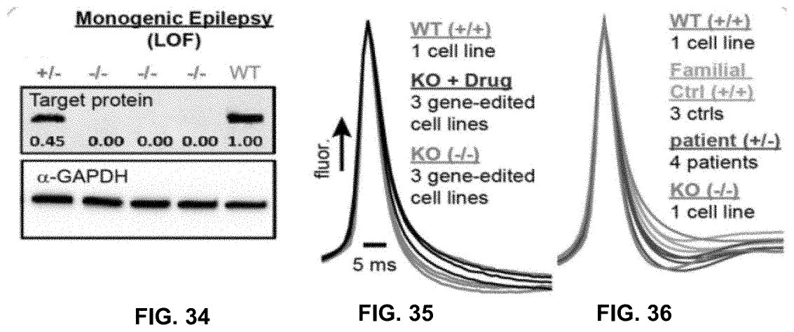

As shown in FIG. 34, the protein that was the target of the knockout was eliminated in the homozygous knockout cells and had reduced expression in the heterozygous knockout cells.

FIG. 35 provides a spike from voltage traces recorded across multiple cell lines that were either wildtype (green), homozygous for the knockout (pink), or homozygous for the knockout and stimulated in the presence of a clinically effective compound (black). As shown, different wildtype cells lines and different knockout cell lines provided consistent spike shapes, with the wildtype and homozygous lines consistently differing from one another. Further, stimulation in the presence of the clinically effective compound consistently moved the spike shape from that of the knockout closer to that of the wildtype.

FIG. 36 provides a spike from voltage traces recorded across multiple cell lines that were either wildtype (green), a homozygous knockout (pink), from a patient with a heterozygous knockout mutation (purple), or from familial controls for the patient lines that did not include a knockout (blue). The heterozygous patient cell lines produced a consistent, but less severe phenotype than the homozygous knockout mutant lines.

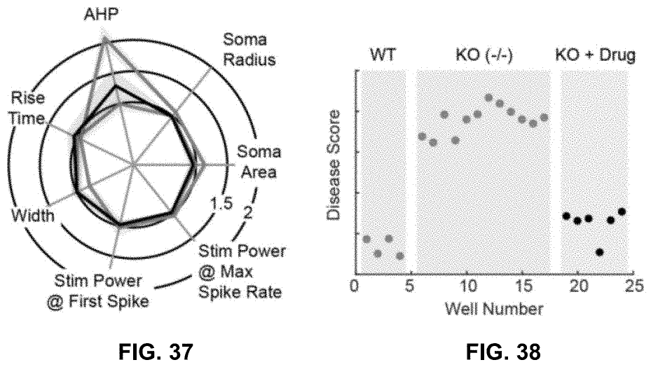

FIG. 37 provides a multidimensional radar plot representative of a functional phenotype generated using a machine learning system from features extracted from the voltage traces that provided the spikes in FIG. 35. The plot that reveals changes in neuronal morphology, action potential shape, and spike train behavior between the wildtype cells (green), the homozygous knockout cells (pink), and the homozygous knockout cells stimulated in the presence of the clinically effective compound (green). As expected, treatment with the clinical compound moves the function phenotype of the homozygous knockout cell lines towards those for the WT for all metrics.

FIG. 38 provides a disease score that represents a further dimensionality reduction of the action potential features to quickly characterize the effects the mutations and the clinically effective compound have on cellular behavior. This disease score provides a robust phenotype that is consistent and comparable across all lines tested. Further, as expected, even in this reduced dimensionality, the methods and systems of the invention can readily determine the ability of the drug to rescue the WT phenotype.

In a related experiment, a CRISPR/Cas9 system was used to introduce a gain-of-function mutation in an ion channel for a monogenic epilepsy disease model.

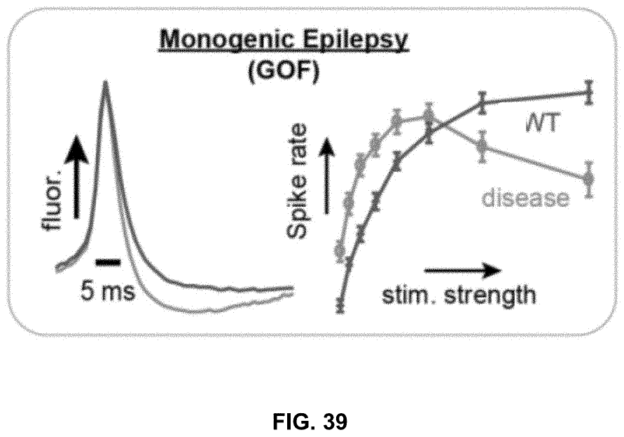

FIG. 39 provides spike parameters and spike rates measured for the gain-of-function cells (blue) and wildtype control cells (purple). As expected, the mutation changes action potential shape and firing behavior between disease model neurons and their isogenic controls.

Thus, in addition to testing diverse pharmacological mechanisms, the systems and methods of the disclosure can be applied to many neuronal types for different disease models. In just the examples provided, the systems and methods of the disclosure were used to record action potential features to develop functional phenotypes characterizing changes in cellular behavior caused by pharmacological effects in rodent CNS neurons, rodent DRG sensory neurons, and multiple types of human iPSC-derived neurons including NGN2 cortical excitatory, inhibitory, and motor neurons. Moreover, the examples include different neurological disease models, including disease models in isogenic backgrounds using gene knock-out or knock-in with CRISPR/Cas9 and with patient-derived neurons.

### Example 6: High-Throughput, Whole-Field Stimulation Assay

In addition to intrinsic excitability measurements described above, the systems and methods of the disclosure can generate incisive measurements into synaptic function. Methods may be used to measure excitatory and inhibitory post-synaptic potentials (EPSPs and IPSPs) in individual cells, information that cannot be obtained with calcium imaging or micro-electrode arrays. Advantageously, the systems and methods can be implemented robustly in 96- and 384-well plates formats with a throughput comparable to that of excitability measurements.

A high-throughput screening of synaptic function was performed with distinct populations of E18 rat hippocampal neurons: pre-synaptic neurons expressing the actuator CheRiff and post-synaptic neurons expressing the voltage-sensor QuasAr using Cre recombinase and floxed constructs. All cells expressed CreOFF-CheRiff (Cre excises CheRiff and turns off expression) and CreON-QuasAr (Cre flips QuasAr to the forward orientation, turning on expression). Cre was added at low titer to transduce subsets of neurons creating disjoint populations of neurons expressing either QuasAr or CheRiff. A brief pulse of blue light was transmitted to the neurons to actuate action potentials in the presynaptic cells, and post-synaptic potentials were detected in postsynaptic cells.

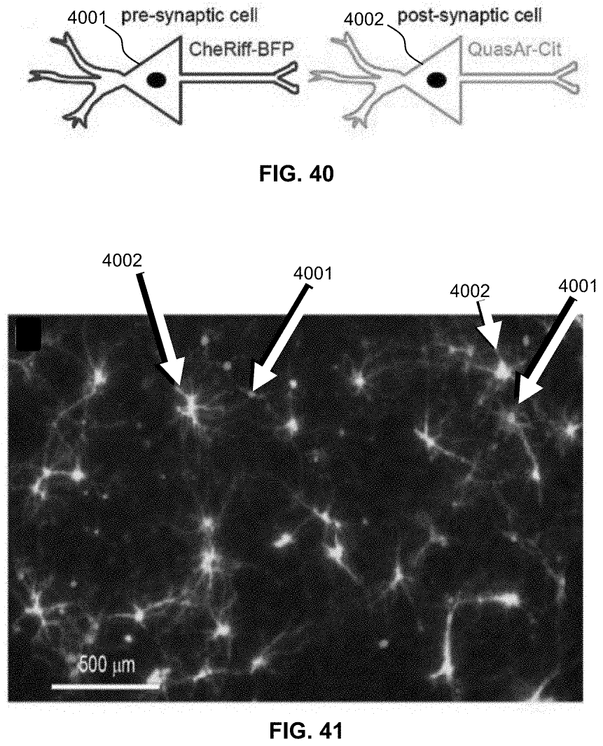

FIG. 40 shows that CheRiff is expressed in a subset of neurons (pre-synaptic neurons 4001) (typically 10-50%) and QuasAr is expressed in the rest (typically 50-90%) (post synaptic neurons 4002).

FIG. 41 provides a fluorescence image obtained using a microscope, as described herein, showing QuasAr fused with citrine (green), CheRiff fused with EBFP2 (blue), and nuclear trafficked TagRFP (red) used for automated image segmentation.

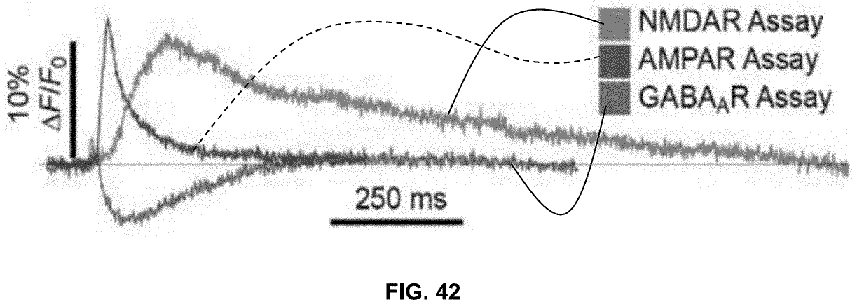

FIG. 42 shows single-cell fluorescent traces showing postsynaptic potentials (PSPs). Synaptic signals were independently probed by pharmacologically isolating AMPA, NMDA and GABA

FIG. 43 shows modulation of single cell PSPs in response to control agonists and blockers for the AMPAR and GABAAR assays. CheRiff stimulation shown at the bottom.

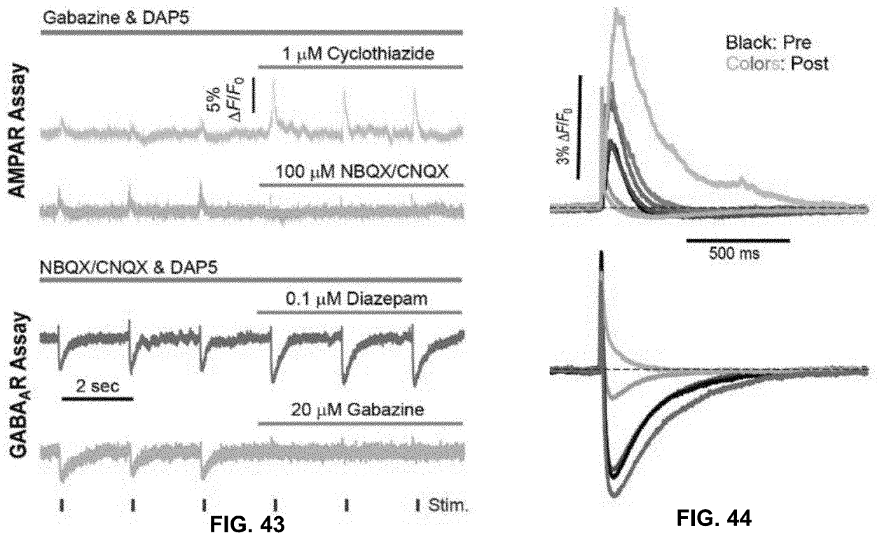

FIG. 44 shows average PSP traces for control pharmacology: Black: pre-drug; Cyan: competitive blocker [AMPAR: 100 μM NBQX/CNQX, 389 cells. GABAAR: 20 μM Gabazine, 176 cells], Green: negative allosteric modulator (NAM) [100 μM GYKI 53655, 291 cells. GABAAR: 30 μM Picrotoxin, 176 cells], Purple: vehicle control [AMPAR: 167 cells. GABAAR: 236 cells], and Blue, Red, & Yellow: positive allosteric modulator (PAM) [AMPAR: 0.1-1 μM Cyclothiazide, 512 cells. GABAAR: 0.1-1 μM Diazepam, 244 cells].

As shown in FIGS. 43-44, using appropriate postsynaptic channel blockers, enables isolation of excitatory, depolarizing voltage changes resulting from AMPA channels and NMDA channels and inhibitory hyperpolarizing voltage changes from GABAA channels.

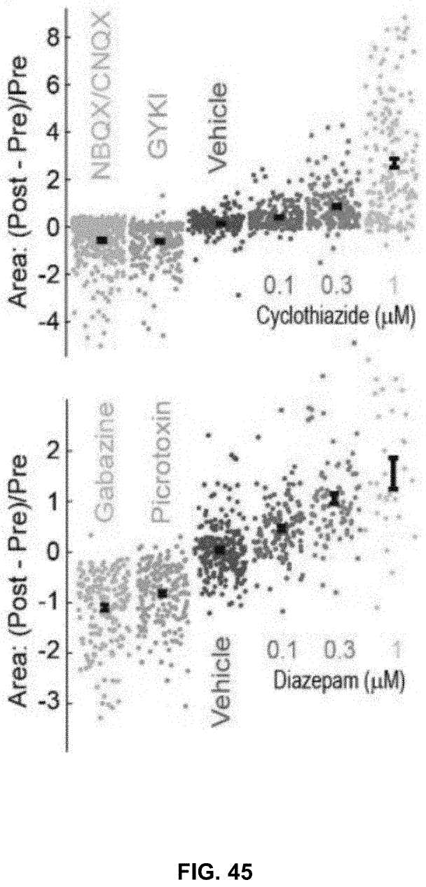

FIG. 45 gives dot-density plots (each dot is one post-synaptic neuron) showing the drug-induced change in PSP area normalized to the mean pre-drug response. Black whiskers are mean±SEM. The density plots highlight the large number of individual cells measured and shows clear effects of both positive and negative channel modulators. Additional insight can be obtained if cell types are identified with a fluorescent label. For example, excitatory and inhibitory cells can be distinguished by transducing cells with a lentiviral construct containing GFP driven by an inhibitory promoter, and excitatory and inhibitory sub-types can be identified using mouse Cre lines. A synaptic assay can resolve individual synapses by stimulating single presynaptic cells with the DMD of the microscope.

### Example 7: High Throughput Screening

The methods and systems of the disclosure can be used to implement high-throughput screening (HTS) of drugs using functional phenotypes derived from action potential features to characterize cellular behavior changes due to diseases and pharmacological compounds.

Production of plates is automated for the drug screening assay to identify the disease associated phenotype and optimized for high-throughput drug screening. Heatmap analysis is used to characterize intraplate and interplate variability. Changes in cell plating and handling, stimulus protocol, and assay duration are tested and result in intraplate and interplate variability <20% while maintaining a Z′ value>0.3 as described.

DMSO tolerance is defined using concentration-response experiments to identify DMSO levels that produce <10% changes in the assay window magnitude compared with buffer control values. Following confirmation of assay readiness, a small set of five screening plates is randomly selected from the library to guide the selection of a final screening concentration. These plates of compounds are screened in duplicate at 1, 3, 7 and 10 μM concentrations. A compound concentration that yields a hit rate of about 1%, with hits defined as a change of greater than 3 standard deviations (SD's) from control values is selected. Using this concentration, a high number of true hits are captured with minimal false positives.

A pilot screen of an FDA approved drug library and tool compounds uses a library of approximately 2400 drugs approved worldwide. That library is screened to find a selected set of available tool compounds at the selected screening concentration. This step serves as a final test of assay readiness for HTS and provides a dataset to establish hit selection criteria, as this library is likely to contain active compounds. Compound libraries are prepared in barcoded 384-well plates in 100% DMSO.

Exemplary methods include production and banking of reagents for HTS. To ensure uniform cell preparation, one may generate, aliquot, and freeze 300 million iPSC-derived NGN2 neurons, 100 million primary rodent glia, and large batches of lentivirus encoding the Optopatch constructs. Each batch is sufficient to execute the screen 1.5 times. Automated cell culture processes are applied throughout HTS activities to improve efficiency and uniformity.

Exemplary methods include HTS screen and hit confirmation. Compounds are screened in 384-well format (n=1) at the screening concentration selected, with 32 wells in each plate reserved for controls. The scan time for each plate depends on the assay protocol, but generally takes approximately 90 minutes, which enables screening of >5,000 compounds/week on one microscope as described herein at 3 screening days/week. Plates with excess variability (Z′<0.3), low number of active cells, or non-uniform plating are flagged for repeat. Hit selection and confirmation are performed following HTS.

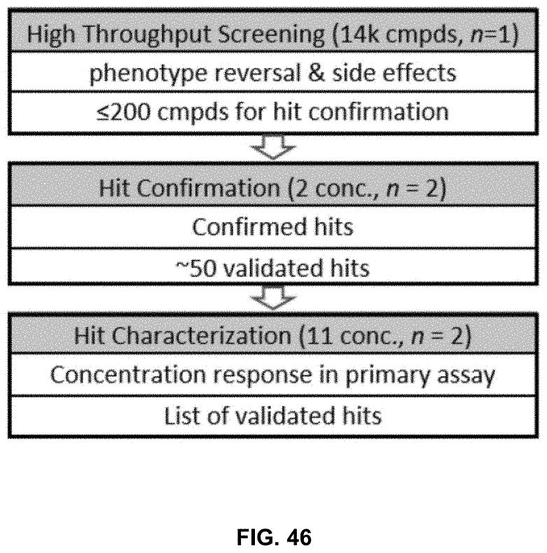

FIG. 46 diagrams an exemplary method for high-throughput screening.

Hits are initially selected based on reversal of the multiparameter phenotype score and side effect score. Hit selection criteria are based on statistical criteria with hits defined as compounds exhibiting >3 SD changes from in-plate control values.

Activity of up to 200 selected hits is first confirmed in duplicate at 1× and 0.3× the screening concentration. 2× concentrations help identify compounds with non-monotonic concentration response. Confirmed hits are tested in 11-pt concentration-response to quantitatively characterize phenotype reversal and side effects. Results confirm platform performance.

### Example 8: Computer System

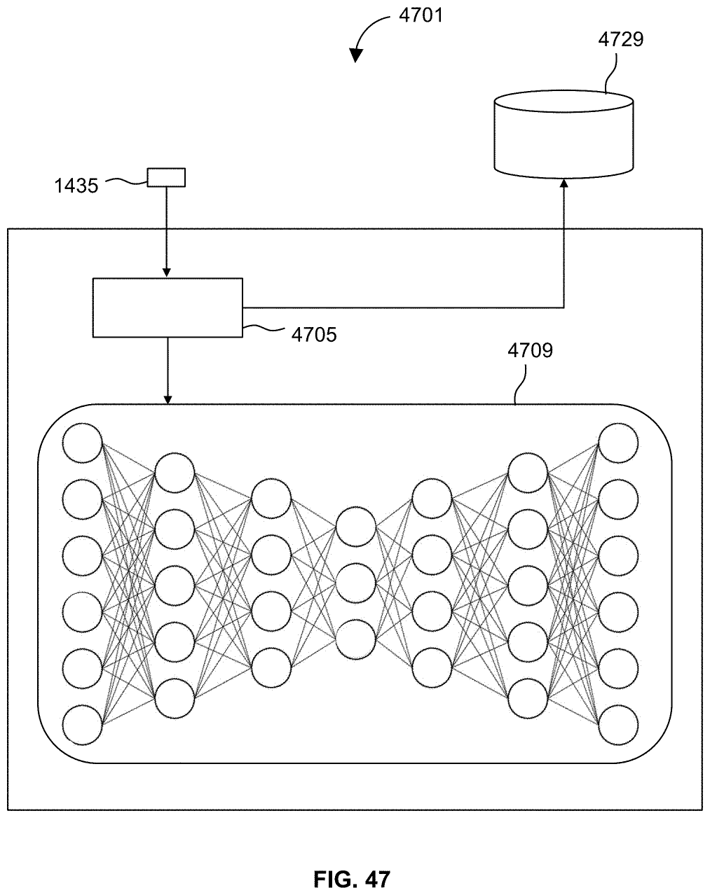

FIG. 47 shows a computer system 4701 makes a recording of activity of one or more electrically-active cells. Video data flows from image sensor 1435 to a processing module 4705 that uses a processor coupled to memory to present the recording to a machine learning system 4709 trained on training data comprising recordings from cells with a known pathology and cells without the pathology. The machine learning system 4709 reports a phenotype of the electrically-active cells. The processing module 4705 may measure features from action potentials within the video data, which features may be presented as inputs to the machine learning system 4709. Optionally, a budget wrapper selects only a limited number (e.g., 8, 10, or 12 or so) of such features to be used as input. The selected data is presented as input to the machine learning system 4709, which gives, as output, a phenotype of the living, electrically active cells being filmed.

Because the output is a phenotype, the output (and thus the machine learning system 4709) reports whether the cells are affected by a pathology. Thus the machine learning system 4709 can show when a test compound is having efficacy on disease-affected cells.

The system 4701 is operable for compressing raw movie data. The processing module may perform the compressing by obtaining digital video data, via sensor 1435, of electrically active cells. The system 4701 processes the video data in a block-wise manner by, for each block, calculating a covariance matrix and an eigenvalue decomposition of that block and truncating the eigenvalue decomposition and retaining only a number of principal components, thereby discarding noise from the block. Further, the system 4701 writes the video to memory as a compressed video using only the retained principal components. In preferred embodiments, the system 4701 compresses the video by a factor of at least ten, preferably even by about 20× to 200× compression, allowing the system 4701 to write the compressed video to a remote storage 4729, which may be a server system, cloud computing resource, or third-party system.

### Example 9: Hierarchical Bootstrapping Algorithm

Embodiments include a hierarchical bootstrapping function with capabilities for statistical tests and confidence interval construction as well as power analysis for hierarchically nested data; and a recursive resampling algorithm that allows to sample from hierarchical data at an arbitrary number of levels. Exclusively focusing on nested data (the relevant and valuable case for the disclosure) enables us to fully leverage this structure and build powerful and efficient custom tools for in vitro biology applications. For measurements from electrically active cells made using a sensor 1435, a processing module 4705 can recursively re-sample the features.

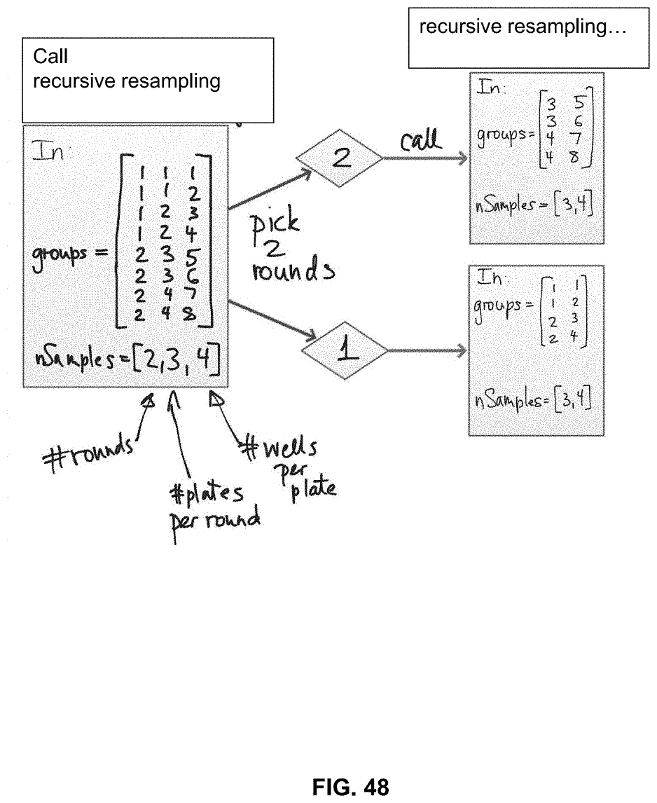

FIG. 48 diagrams a recursive resampling routine that can be called by the processing module 4705. The main inputs include a table with metadata defining hierarchy and features to use; a list of columns containing the hierarchy information (e.g. {‘CellId’, ‘WellId’}); a number of samples to choose per level (if not specified by user, algorithm emulates the size of the original dataset, e.g., if data consists of 2 rounds of 6 plates with 96 wells each, will sample 2 rounds with 6 plates with 96 wells each); and an estimator to use and significance level. The routine may optionally include features to compute statistic on (if none provided, will use all numeric features in table) and/or a column specifying populations (currently support for 1 or 2 populations).

Generally, preprocessing may include extracting a matrix of desired numerical features to perform statistics on and, using hierarchy information, preparing grouping information and inputs to a resampling function. If performing power analysis: add signal of specified size to true measurement noise data. For a desired number of iterations: sample row indices, use row indices to access feature matrix and resample all features at once, compute desired test statistics for all features at once, and prepare result table based on desired estimator. The routine outputs a result table containing desired estimate, table of statistics computed each iteration. The implementation of the resampling algorithm accommodates an arbitrary number of sampling levels due to a recursive implementation. Main inputs include a matrix of hierarchy group information (optionally containing extra column with population information) and a Numbers of samples to pick per level (if all zeros, infers sample sizes from group information and returns sample of same format). Output: vector of resampled row indices.

As an example for first resampling step and recursive call, the routine will sample a desired number at highest level (taking into account population information if provided). For each sample, the routine selects the corresponding lower hierarchy levels and call algorithm on lower-level data. Sample indices are combined into one output vector containing the sampled row indices from the original table.

The described recursive bootstrapping algorithm is useful for performing power analyses. A power analysis may be useful for determining on what scale an experiment must be performed (number of wells, replicates, tests, etc.) for a given biological or chemical query.

Another embodiment uses a preferably non-recursive bootstrapping algorithm to create augmented data useful when training a machine learning system 4709 to avoid the trained machine learning system 4709 overfitting the data.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof.

What is claimed is:

1. A method for characterizing cellular activity, the method comprising:
making a recording of activity of one or more electrically-active cells;
presenting the recording to a machine learning system trained on training data comprising recordings from cells with a known pathology and cells without the pathology; and
reporting, by the machine learning system, a phenotype of the electrically-active cells.

2. The method of claim 1, wherein the recording comprises one or more action potentials exhibited by the electrically-active cells.

3. The method of claim 1, wherein the machine learning system reports the phenotype of the electrically-active cells as having, or not having, the pathology.

4. The method of claim 1, further comprising exposing the electrically-active cells to a test compound.

5. The method of claim 1, wherein the machine learning system reports the phenotype of the electrically active cells as reverting from having the pathology to not having the pathology with exposure to test compound.

6. The method of claim 1, wherein the recording is a digital movie made by imaging the electrically-active cells through a microscope with a CMOS images sensor.

7. The method of claim 1, wherein the machine learning system is resident in a computer system comprising a processor coupled to memory, and the recording is saved in the memory.

8. The method of claim 1, wherein the recording captures action potentials, and the method further comprises measuring, and storing, a plurality of features from the action potentials.

9. The method of claim 1, further comprising measuring features from the recording and presenting the features to the machine learning system, optionally wherein the features comprise one or more of spike rate, spike height, spike width, depth of afterhyperpolarization, onset timing, timing of cessation of firing, inter-spike interval, adaptation over a constant stimulation, a first derivative of spike waveform, and a second derivative of spike waveform.

10. The method of claim 1, further comprising operating the machine learning system under control of a budget wrapper that limits a number of features that are presented to the machine learning system.

11. The method of claim 1, further comprising extracting greater than 100 features from the recording and further wherein a budget wrapper presents fewer than about 20 of the features to the machine learning system.

12. The method of claim 1, wherein the machine learning system comprises a neural network.

13. The method of claim 12, wherein the neural network is an autoencoder neural network that operates by representation learning.

14. The method of claim 13, wherein the autoencoder has been trained using manually selected training data comprising the recordings from cells with the known pathology and the cells without the pathology in samples that have been exposed to known compounds with known efficacy and control samples that have not be exposed to the known compounds.

15. The method of claim 1, wherein the machine learning system was trained using a hierarchical bootstrapping algorithm.

16. The method of claim 15, wherein the hierarchical bootstrapping algorithm recursively samples from an arbitrary number of levels of nested data.

17. The method of claim 15, wherein the hierarchical bootstrapping algorithm creates augmented samples by re-sampling with replacement from features measured from action potentials in the training data, and wherein the machine learning system is trained using the augmented samples.

18. A method for compressing raw movie data, the method comprising:
obtaining digital video data of electrically active cells;
processing the video data in a block-wise manner by, for each block, calculating a covariance matrix and an eigenvalue decomposition of that block and truncating the eigenvalue decomposition and retaining only a number of principal components, thereby discarding noise from the block, and
writing the video to memory as a compressed video using only the retained principal components.

19. The method of claim 18, wherein the blocks are selected by parcellating the data using region-based tiling based on a local intensity maxima of a mean movie frame.

20. The method of claim 18, wherein the digital video data is obtained from the electrically active cells expressing optical reporters of cellular electrical activity.

21. The method of claim 18, wherein the cells are neurons and the digital video data shows action potentials propagating along axons of the neurons.

22. The method of claim 21, wherein the compressed video can be retrieved and played to display the action potentials propagating along the axons of the neurons.

23. The method of claim 21, further comprising measuring, by a machine learning system, features from the action potentials, wherein the machine learning system obtains the same values for the measured features whether measuring from the digital video data or the compressed video.

24. The method of claim 23, wherein the features comprise one or more of voltage, fluorescence versus time, spike height, spike width, shape change, slope, frequency, and timing.

25. The method of claim 18, wherein the compressed video occupies less than about 10% of disc space required for the digital video data.

26. The method of claim 18, wherein the obtaining step comprises filming, through a microscope and using a digital image sensor, live neurons firing.

27. The method of claim 26, wherein the digital image sensor is connected to a computer that performs the processing step, and further wherein the compressed video is written to a remote computer via an Internet connection.

28. The method of claim 26, wherein the digital image sensor produces over 50 terabytes of the digital video data in one day.

29. The method of claim 28, wherein the processing step compresses the digital video data by at least about 20×.
